(19) United States
(12) Patent Application Publication
(10) Pub. No.: US 2024/0238306 A1
(43) Pub. Date: Jul. 18, 2024

(54) TREATING INFLUENZA USING SUBSTITUTED POLYCYCLIC PYRIDONE DERIVATIVES AND PRODRUGS THEREOF IN A SUBJECT HAVING INFLUENZA AND A COMPLICATION RISK FACTOR

(71) Applicant: Shionogi & Co., Ltd., Osaka (JP)

(72) Inventors: Takeki UEHARA, Osaka (JP); Keiko KAWAGUCHI, Osaka (JP); Ann HOWELL, Florham Park, NJ (US); Simon PORTSMOUTH, Florham Park, NJ (US)

(21) Appl. No.: 18/525,177

(22) Filed: Nov. 30, 2023

Related U.S. Application Data
(63) Continuation of application No. 18/152,449, filed on Jan. 10, 2023, now abandoned.
Application No. 18/152,449 is a continuation of application No. 16/814,669, filed on Mar. 10, 2020, now abandoned.

(30) Foreign Application Priority Data
Mar. 12, 2019 (WO) PCT/IB2019/052012

(51) Int. Cl. A61K 31/5383; A61P 31/16

(52) U.S. Cl. CPC A61K 31/5383; A61P 31/16

(57) ABSTRACT

A method for treating an influenza virus infection is described. The disclosed method generally involves administering an effective amount of a compound, for example baloxavir marboxil, to a subject having an influenza virus infection and at least one complication risk factor. Generally, the amount is effective such that a reduction in a time to improvement of at least one symptom of an influenza virus infection is statistically significant as compared to that of a non-treated subject.

### FIELD

The present disclosure relates generally to treating an influenza virus infection in a subject having an influenza virus infection and a complication risk factor, using a substituted polycyclic pyridone derivative having cap-dependent endonuclease inhibitory activity, a prodrug thereof, and a pharmaceutical composition including the same.

### BACKGROUND

Influenza causes considerable morbidity and mortality with the greatest incidence of influenza-related complications, hospitalization and death in high risk groups including persons ≥50 years of age and those with underlying medical conditions (Non-Patent Documents 1-2). Until recently, treatment of influenza has been limited to 2 classes of antiviral medication. Widespread resistance to M2 ion-channel inhibitors and emergence of resistance to neuramindase inhibitors (NAIs), especially oseltamivir, in treated patients and in community clusters, including global circulation of oseltamivir-resistant seasonal influenza type A(H1N1) in 2008-2009, highlight the need for new agents with different mechanisms of antiviral action (Non-Patent Documents 3-6). Further, oseltamivir is less active in vitro for influenza type B than type A viruses and appears to be less effective in treating influenza type B than type A virus infections (Non-Patent Documents 7-8). While observational studies, some including high risk patients, have found that timely oseltamivir therapy is associated with reduced risks of influenza-associated pneumonia, hospitalization, mortality, and cardiovascular events, few randomized, placebo-controlled trials (RCTs) of NAIs in high-risk patients have been published (Non-Patent Documents 9-14).

Several new influenza antivirals that target different protein subunits of the influenza polymerase complex are undergoing clinical studies (Non-Patent Document 15). Baloxavir marboxil (BXM) is the small-molecule prodrug of baloxavir that has antiviral activity against influenza type A and type B viruses, including those resistant to current antivirals (Non-Patent Document 16). BXM was recently approved for treatment of uncomplicated influenza in otherwise healthy individuals ≥12 years old. BXM was associated with more rapid reductions in infectious virus titers than placebo or oseltamivir (Non-Patent Document 17). Therefore, it is desired to expand application of BXM to treatment in adult and adolescent outpatients with acute influenza who are at high risk of influenza-related complications.

Patent Document 1-6 describe BXM and/or compounds having similar structures to substituted polycyclic pyridone derivatives.
Patent Document 1: WO2010/147068
Patent Document 2: WO2012/039414
Patent Document 3: WO2016/175224
Patent Document 4: WO2017/104691
Patent Document 5: WO2017/221869
Patent Document 6: WO2018/030463
Non-Patent Document 1: Grohskopf L A, Sokolow L Z, Broder K R, Walter E B, Fry A M, Jernigan D B. Prevention and Control of Seasonal Influenza with Vaccines: Recommendations of the Advisory Committee on Immunization Practices-United States, 2018-19 Influenza Season. MMWR Recomm Rep 2018;67:1-20
Non-Patent Document 2: Molinari N A, Ortega-Sanchez I R, Messonnier M L, et al. The annual impact of seasonal influenza in the US: measuring disease burden and costs. Vaccine 2007;25:5086-96
Non-Patent Document 3: Memoli M J, Athota R, Reed S, et al. The natural history of influenza infection in the severely immunocompromised vs nonimmunocompromised hosts. 2014;58:214-24
Non-Patent Document 4: Hurt A C, Chotpitayasunondh T, Cox N J, et al. Antiviral resistance during the 2009 influenza type A H1N1 pandemic: public health, laboratory, and clinical perspectives. Lancet Infect Dis 2012; 12:240-8
Non-Patent Document 5: Li T C, Chan M C, Lee N. Clinical Implications of Antiviral Resistance in Influenza. Viruses 2015;7:4929-44

Non-Patent Document 6: Hu Y, Lu S, Song Z, et al. Association between adverse clinical outcome in human disease caused by novel influenza type A H7N9 virus and sustained viral shedding and emergence of antiviral resistance. 2013;381:2273-9
Non-Patent Document 7: Lee N, Hui D S, Zuo Z, et al. A prospective intervention study on higher-dose oseltamivir treatment in adults hospitalized with influenza type A and type B infections. Clin Infect Dis 2013;57:1511-9
Non-Patent Document 8: Sugaya N, Mitamura K, Yamazaki M, et al. Lower clinical effectiveness of oseltamivir against influenza type B contrasted with influenza type A infection in children. Clin Infect Dis 2007;44:197-202
Non-Patent Document 9: Madjid M, Curkendall S, Blumentals W A. The influence of oseltamivir treatment on the risk of stroke after influenza infection. Cardiology 2009; 113:98-107
Non-Patent Document 10: Dobson J, Whitley R J, Pocock S, Monto A S. Oseltamivir treatment for influenza in adults: a meta-analysis of randomised controlled trials. Lancet 2015;385:1729-37
Non-Patent Document 11: Lalezari J, Campion K, Keene O, Silagy C. Zanamivir for the treatment of influenza type A and type B infection in high-risk patients: a pooled analysis of randomized controlled trials. Arch Intern Med 2001; 161:212-7
Non-Patent Document 12: Murphy K R, Evindson A, Pauksens K, et al. Efficacy and Safety of Inhaled Zanamivir for the Treatment of Influenza in Patients with Asthma or Chronic Obstructive Pulmonary Disease: A Double-Blind, Randomised, Placebo-Controlled, Multicentre Study. Clin Drug Invest 2000;20:337-49
Non-Patent Document 13: Johnston S L, Ferrero F, Garcia M L, Dutkowski R. Oral oseltamivir improves pulmonary function and reduces exacerbation frequency for influenza-infected children with asthma. Pediatr Infect Dis J 2005;24:225-32
Non-Patent Document 14: Kaiser L, Wat C, Mills T, Mahoney P, Ward P, Hayden F. Impact of oseltamivir treatment on influenza-related lower respiratory tract complications and hospitalizations. Arch Intern Med 2003;163:1667-72
Non-Patent Document 15: McKimm-Breschkin JL, Jiang S, Hui DS, Beigel J H, Govorkova EA, Lee N. Prevention and treatment of respiratory viral infections: Presentations on antivirals, traditional therapies and host-directed interventions at the 5th ISIRV Antiviral Group conference. Antiviral Res 2018; 149:118-42
Non-Patent Document 16: Uehara T, Shishido T, Ishibashi T, et al. S-033188, a Small Molecule Inhibitor of Cap-dependent Endonuclease of Influenza type A and type B Virus, Leads to Rapid and Profound Viral Load Reduction. Options IXfor the Control of Influenza Chicago, Illinois2016
Non-Patent Document 17: Hayden F G, Sugaya N, Hirotsu N, et al. Baloxavir Marboxil for Uncomplicated Influenza in Adults and Adolescents. N Engl J Med 2018;379:913-23

### SUMMARY

A method for treating an influenza virus infection is described. The disclosed method generally involves administering an effective amount of a compound to a subject in order to treat the influenza virus infection, where the subject has (1) an influenza virus infection, and (2) at least one complication risk factor. In one example, the amount of the compound administered is effective such that reduction of the time to improvement of at least one symptom of an influenza virus infection in the subject as compared to that of a non-treated subject is statistically significant. In one example, the amount of the compound administered is effective such that avoidance and/or reduction in the incidence of an influenza-related complication in the subject as compared to that of a non-treated subject is statistically significant. In one example, the amount of the compound administered is effective such that reduction of the time to cessation of viral shedding by a virus titer in the subject as compared to that of a non-treated subject is statistically significant. In one example, the amount of the compound administered is effective such that reduction of the virus titer count in the subject as compared to that of a non-treated subject is statistically significant. The non-treated subject is a subject that has not been administered the compound.

In one example, the compound has one of the following formulae:

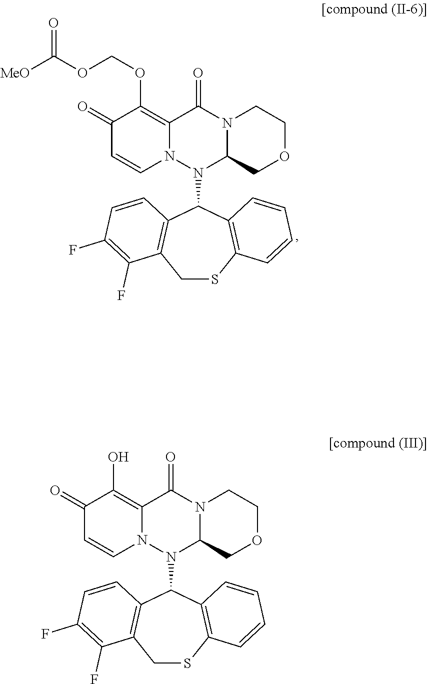 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof.

In one example, a p-value indicating the statistical significance of the reduction of the time to improvement of at least one symptom of the influenza virus infection is less than 0.05, alternately less than 0.005, preferably less than 0.05. In one example, a p-value indicating the statistical significance of the avoidance and/or reduction in an incidence of an influenza-related complication is less than 0.05, alternately less than 0.005, preferably less than 0.05. In one example, a p-value indicating the statistical significance of the reduction of the time to cessation of viral shedding by a virus titer is less than 0.05, alternately less than 0.005, preferably less than 0.05. In one example, a p-value indicating the statistical significance of the reduction of a virus titer count is less than 0.05, alternately less than 0.005, preferably less than 0.05.

In general, a subject with a complication risk factor for an influenza virus infection is considered to be high risk of an influenza complication due to the presence of a certain criteria. In one example, the complication risk factor can include one or more of chronic lung diseases, endocrine disorders, being an age that is 65 or older, a current resident of a long-term care facility, metabolic disorders, a compromised immune system, neurological disorders, neurodevelopmental disorders, heart diseases, blood disorders, being a female who is within two weeks postpartum and is not breastfeeding, having American Indian or Alaskan native heritage, and morbid obesity. A more preferred example, the complication risk factor can include one or more of chronic lung diseases, endocrine disorders, being an age that is 65 or older, heart diseases and morbid obesity.

In one example, an influenza-related complication can include death, hospitalization, or one or more of disorder selected from the group consisting of sinusitis, otitis media, bronchitis and pneumonia.

In one example, the influenza virus is a type B influenza virus.

In one example, the time to improvement of at least one symptom is a time from the initial administration of the compound to an improvement of at least one of the symptoms of an influenza virus infection (influenza symptoms), as compared to the respective symptoms before the administration of the compound, where the improvement of at least one symptom lasts for at least 21.5 hours. In one example, at least one of the influenza symptoms is improved within at most 86 hours from the first administration of the compound, as compared to the respective symptoms before the administration of the compound.

In one example, at least one of the influenza symptoms is a systemic symptom or a respiratory symptom. In one example, a systemic symptom includes one or more symptoms of headache, feverishness, chills, muscular pain, joint pain, and fatigue. In one example, a respiratory symptom includes one or more symptoms of coughing, sore throat, and nasal congestion.

In some examples, the virus titer in the treated subject is reduced by at least about 2.8 log₁₀ TCID₅₀/mL, alternately at least about 3.3 log₁₀ TCID₅₀/mL, relative to that of when the compound is first administered to the subject. In one example, the reduction in the virus titer is measured on Day 2 after the compound is first administered to the subject. “Day 2” means one day after the compound is first administered to the subject.

In one example, the number of times the compound is administered is not particularly limited. In another example, the compound can be administered only once. In another example, the compound can be administered only two times. In another example, the compound can be administered only three times.

In one example, the effective amount of the compound is in a range from at or about 0.1 to at or about 3000 mg. In another example, the effective amount of the compound is in a range from about at or 0.1 to at or about 240 mg. In another example, the effective amount of the compound is in a range from about at or 3 to at or about 80 mg. In yet another example, the effective amount of the compound is in a range from at or about 40 to at or about 80 mg. In yet another example, the effective amount is in a range from at or about 3 to at or about 80 mg per dose. In yet another example, the effective amount is in a range from at or about 10 to at or about 80 mg per dose. In a more preferred example, the effective amount of the compound is in a range from at or about 40 to at or about 80 mg per dose.

In one example, the compound is administered based on the weight of the subject. In one example, the compound can be administered as a weight-based dose. In one example, about 40 mg is administered to a subject weighing about 40kg to less than about 80 kg. In one example, about 80 mg is administered to a subject weighing above at or above 80 kg.

### Aspects

1. A method for treating an influenza virus infection, comprising:
administering an effective amount of a compound to a subject having (1) an influenza virus infection, and (2) a complication risk factor,
wherein the compound has one of the following formulae:

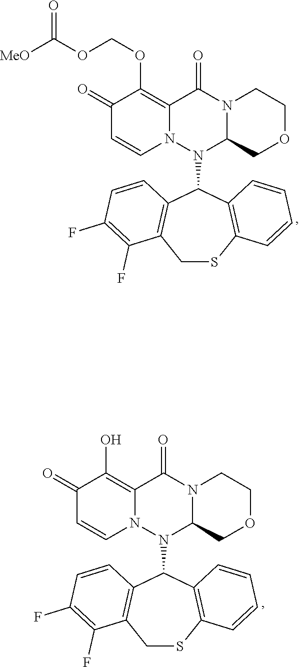 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof.

2. The method of aspect 1, wherein the subject is a subject that has been symptomatic for no more than 48 hours.

3. The method of aspect 1 or 2, wherein the effective amount administered to the subject is an amount with which at least one of the following (i)-(iv) occurs in the subject compared to that of a non-treated subject:
(i) reduction of a time to improvement of at least one symptom of the influenza virus infection,
(ii) avoidance and/or reduction of an influenza-related complication,
(iii) reduction of a time to cessation of viral shedding by a virus titer, and
(iv) reduction of a virus titer.

4. The method of aspect 3, wherein (i) reduction of the time to improvement of at least one symptom of the influenza virus infection is statistically significant as compared to that of a non-treated subject, (ii) avoidance and/or reduction of the influenza-related complication is statistically significant as compared to that of a non-treated subject, (iii) reduction of the time to cessation of viral shedding by a virus titer is statistically significant as compared to that of a non-treated subject, and (iv) reduction of the virus titer is statistically significant as compared to that of a non-treated subject.

5. The method of aspect 4, wherein a p-value indicating the statistical significance is less than 0.05.

6. The method of any one of aspects 1 to 5, wherein the complication risk factor is at least one factor selected from the group consisting of a chronic lung disease, an endocrine disorder, being an age that is 65 or older , a current resident of a long-term care facility, a metabolic disorder, a compromised immune system, a neurological disorder, a neurodevelopmental disorder, a heart disease, a blood disorder, being a female who is within two weeks postpartum and is not breastfeeding, having American Indian or Alaskan native heritage, and morbid obesity.

7. The method of aspect 6, wherein the complication risk factor is a chronic lung disease.

8. The method of any one of aspects 3 to 7, wherein the influenza-related complication is at least one complication selected from the group consisting of death, hospitalization, sinusitis, otitis media, bronchitis and pneumonia.

9. The method of aspect 8, wherein the influenza-related complication is sinusitis.

10. The method of aspect 8, wherein the influenza-related complication is bronchitis.

11. The method of any one of aspects 1 to 10, wherein influenza virus causing the influenza virus infection is a type B influenza virus.

12. The method of any one of aspects 3 to 11, wherein the virus titer in the treated subject is reduced by at least about 2.8 log₁₀ TCID₅₀/mL 24 hours after the compound is first administered to the subject relative to that of when the compound is first administered to the subject.

13. The method of any one of aspects 3 to 11, wherein the virus titer in the treated subject is reduced by at least about 3.3 log₁₀ TCID₅₀/mL 24 hours after the compound is first administered to the subject relative to that of when the compound is first administered to the subject.

14. The method of any one of aspects 3 to 13, wherein the time to improvement of symptoms is the time from the initial administration of the compound to an improvement of influenza symptoms, as compared to the respective symptoms before the administration of the compound, and wherein the improvement of symptoms lasts for at least 21.5 hours.

15. The method of any one of aspects 1 to 14, wherein the at least one influenza symptom is improved within at least 24 hours from administration of the effective amount of the compound.

16. The method of any one of aspects 1 to 14, wherein the at least one influenza symptom is improved within at least 48 hours from administration of the effective amount of the compound.

17. The method of any one of aspects 1 to 14, wherein the at least one influenza symptom is improved within at least 72 hours from administration of the effective amount of the compound.

18. The method of any one of aspects 1 to 14, wherein the at least one influenza symptom is improved within at least 86 hours from administration of the effective amount of the compound.

19. The method of any one of aspects 1 to 14, wherein the influenza symptoms are improved within at most 86 hours as compared to the respective symptoms before the administration of the compound.

20. The method of any one of aspects 1 to 19, wherein the effective amount of the compound is in a range from about 0.1 to about 240 mg.

21. The method of any one of aspects 1 to 20, wherein the effective amount of the compound is in a range from about 3 to about 80 mg.

22. The method of aspect 1 to 21, wherein the subject has a weight of 40 kg to less than 80 kg and the dose is about 40 mg, or the subject has a weight of at least 80 kg and the dose is about 80 mg.

23. The method of any one of aspects 1 to 22, wherein the compound is administered only one time.

24. The method of any one of aspects 1 to 23, wherein the compound is administered orally or parenterally.

25. The method of any one of aspects 1 to 24, wherein the at least one symptom is at least one of a systemic symptom and a respiratory symptom.

26. The method of aspect 25, wherein the symptom is the systemic symptom and the systemic symptom includes at least one of headache, feverishness, chills, muscular pain, joint pain, and fatigue.

27. The method of aspect 25, wherein the symptom is the respiratory symptom and the respiratory symptom includes at least one selected from the group consisting of coughing, sore throat, and nasal congestion.

28. A method for treating influenza, comprising: reading a dosage instruction on a package insert or in a package for a pharmaceutical formulation comprising a compound having one of the following formulae:

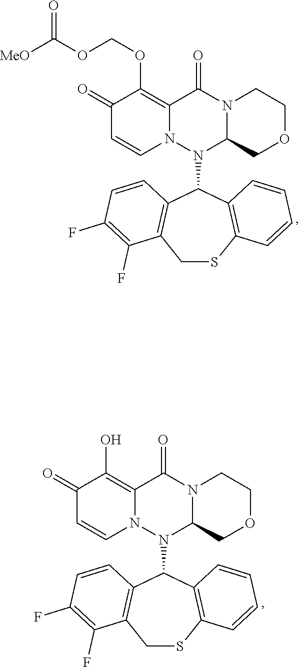 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof; and administering an effective amount of the compound to a subject having:
(1) an influenza virus infection, and
(2) a complication risk factor, in accordance with the dosage instruction,
wherein the amount administered is effective in the subject such that at least one of the following occurs as compared to that of a non-treated subject:
(i) reduction of the time to improvement of at least one symptom of the influenza virus infection is statistically significant,
(ii) avoidance and/or reduction of an influenza-related complication is statistically significant,
(iii) reduction of the time to cessation of viral shedding by a virus titer is statistically significant, and
(iv) reduction of a virus titer is statistically significant.

29. A use of a compound having one of the following formulae:

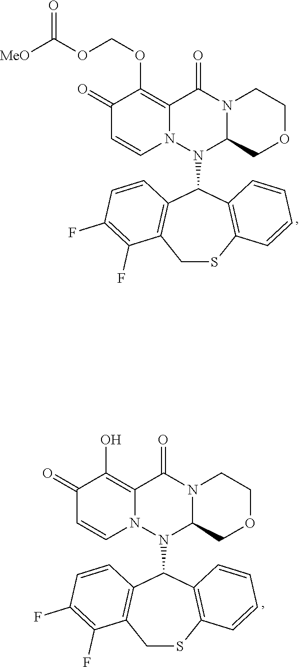 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof, for preparation of a medicament for treating a subject having an influenza virus, wherein the treatment includes administering an effective amount of the compound to a subject having:
(1) an influenza virus infection, and
(2) a complication risk factor, wherein the amount administered is effective in the subject such that at least one of the following occurs as compared to that of a non-treated subject:
(i) reduction of the time to improvement of at least one symptom of the influenza virus infection is statistically significant,
(ii) avoidance and/or reduction of an influenza-related complication is statistically significant,
(iii) reduction of the time to cessation of viral shedding by a virus titer is statistically significant, and
(iv) reduction of a virus titer is statistically significant.

30. A pharmaceutical composition useful for treating a subject having:
(1) an influenza virus infection, and
(2) a complication risk factor, wherein the treatment comprises administering an effective amount of a compound to the subject, wherein the amount administered is effective in the subject such that at least one of the following occurs as compared to that of a non-treated subject:
(i) reduction of the time to improvement of at least one symptom of the influenza virus infection is statistically significant,
(ii) avoidance and/or reduction of an influenza-related complication is statistically significant,
(iii) reduction of the time to cessation of viral shedding by a virus titer is statistically significant, and
(iv) reduction of a virus titer is statistically significant, and
wherein the compound has one of the following formulae:

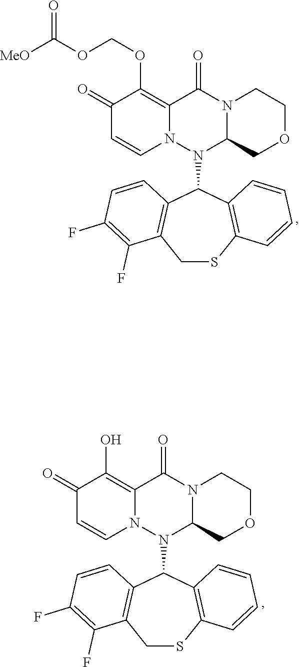 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof.

31. A package, comprising a pharmaceutical formulation comprising a compound having one of the following formulae:

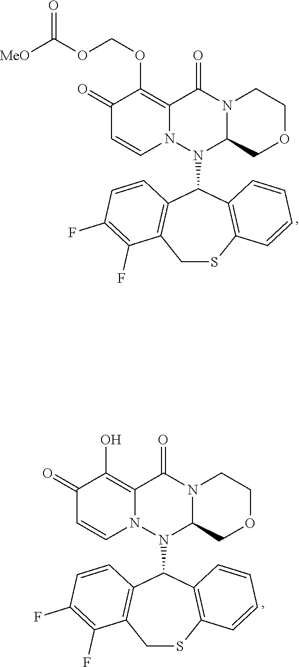 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof; and a dosage instruction on a package insert or in a package for administering an effective amount of the compound to a subject having:
(1) an influenza virus infection, and
(2) a complication risk factor,
wherein the amount administered is effective in the subject such that at least one of the following occurs as compared to that of a non-treated subject:
(i) reduction of the time to improvement of at least one symptom of the influenza virus infection is statistically significant,
(ii) avoidance and/or reduction of an influenza-related complication is statistically significant,
(iii) reduction of the time to cessation of viral shedding by a virus titer is statistically significant, and
(iv) reduction of a virus titer is statistically significant.

### BRIEF DESCRIPTION OF THE DRAWINGS

FIG. 1 is a graph showing the experimental results of the changes in plasma concentration of compound III (baloxavir or “BXA”) where different amounts of prodrug compound II-6 (baloxavir marboxyl or “BXM”) were administered to rats under non-fasting conditions.

FIG. 2 is a table showing the experimental results of measuring the plasma concentration of BXM, after oral administration to rats under non-fasting conditions.

FIG. 3 is a table showing a summary of results of a time to improvement of symptoms of an influenza virus infection in a clinical trial where subjects were randomized to receive a single oral dose of 40 mg or 80 mg of BXM according to body weight, oseltamivir 75 mg twice daily for 5 days, or placebo.

FIG. 4 is a Kaplan-Meier curve of the results of a time to improvement of symptoms in the clinical trial of FIG. 3.

FIG. 5 is a table showing a summary of results of a time to improvement of symptoms in the clinical trial of FIG. 3 for patients having influenza virus type B.

FIG. 6 is a Kaplan-Meier curve of the results of a time to improvement of symptoms for patients in the clinical trial of FIG. 3 having influenza virus type B.

FIG. 7 is a table showing the results of a time to improvement of symptoms for patients having a certain complication risk factor in the clinical trial of FIG. 3.

FIG. 8 is a table showing the results of incidences of influenza-related complications in the clinical trial of FIG. 3.

FIG. 9 is a table showing the results of the time to cessation of viral shedding by a virus titer in the clinical trial of FIG. 3.

FIG. 10 is a table showing a summary of the statistical results of a change from baseline in an influenza virus titer [log10(TCID50/mL)] by time in the clinical trial of FIG. 3.

FIG. 11 is a table showing a summary of the statistical results of a change from baseline in an influenza virus titer [log10(TCID50/mL)] by time for patients having influenza virus type B in the clinical trial of FIG. 3.

### DETAILED DESCRIPTION

A method for treating an influenza virus infection is described. It has been surprisingly discovered that influenza virus infection can be treated in patients with at least one complication risk factor using the compounds disclosed herein, including Compound II-6 and Compound III. In one example, the amount of the compound administered is effective such that at least one of reduction of a time to improvement of at least one symptom of the influenza virus infection, reduction in an incidence of an influenza-related complication, a time to cessation of viral shedding by a virus titer, and reduction of a virus titer count, in the subject is statistically significant.

Generally, the compound that can be used in the disclosed is described as follows.

(1) A compound represented by the following formula (I):

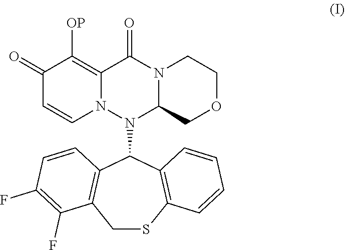

wherein P is hydrogen or a group to form a prodrug, or its pharmaceutically acceptable salt.
(2) The compound according to (1), or its pharmaceutically acceptable salt, wherein the group to form a prodrug is a group selected from the following formula:

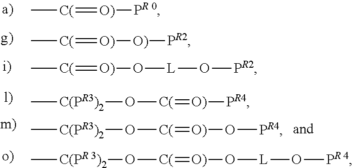

wherein L is straight or branched lower alkylene;
P^{R0} is alkyl;
P^{R2} is alkyl;
P^{R3} is each independently hydrogen; and
P^{R4} is alkyl.

In one example, the compound that can be used in the disclosed method has a formula:

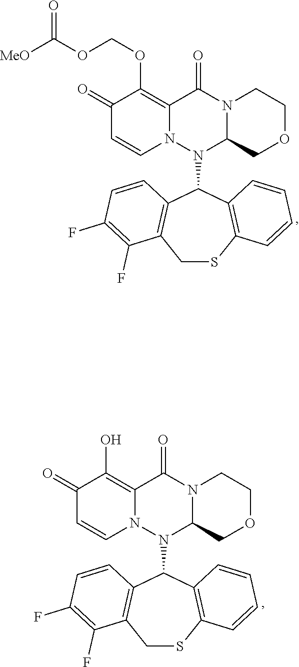 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or its pharmaceutically acceptable salt.

The meaning of various terms used in the present description is explained below. Each term is used in a unified sense, and is used in the same sense when used alone, or when used in combination of other term.

The term of “consisting of” means having only the recited components.

The term of “comprising” means not restricted to only the recited components and not excluding undescribed factors.

The term “high risk patient” refers to a patient who is infected with an influenza virus and who also has a complication risk factor.

The term “complication risk factor” refers to at least one condition selected from the group consisting of chronic lung diseases, endocrine disorders, being an age that is 65 or older, a current resident of a long-term care facility, metabolic disorders, a compromised immune system, neurological disorders, neurodevelopmental disorders, heart diseases, blood disorders, being a female who is within two weeks postpartum and is not breastfeeding, having American Indian or Alaskan native heritage, and morbid obesity. A more preferred example, the complication risk factor can include one or more of chronic lung diseases, endocrine disorders, being an age that is 65 or older, heart diseases and morbid obesity

“Prodrug” in the present description refers to a compound represented by formula (II) in the following reaction formula:

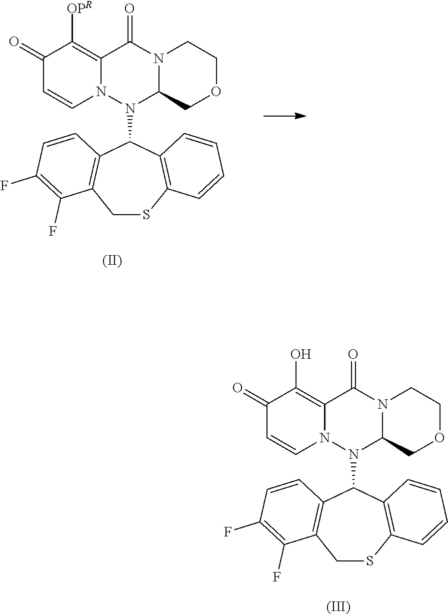

wherein P^{R} is a group to form a prodrug, or its pharmaceutically acceptable salt.

“Group to form a prodrug” in the present description refers to a “P^{R}” group in the formula (II), in the following reaction formula:

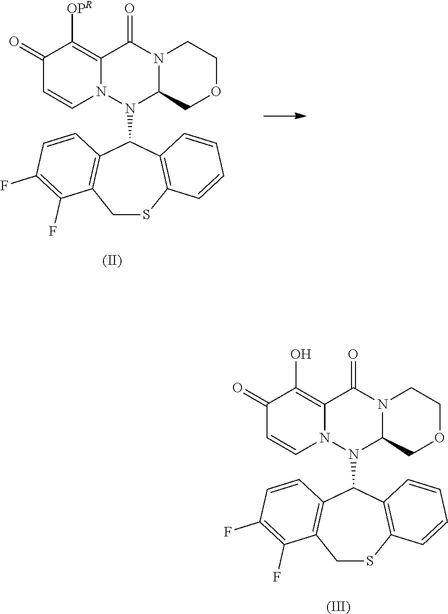

wherein P^{R} is selected from the group consisting of:

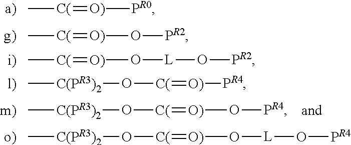

wherein L is straight or branched lower alkylene;
P^{R0} is alkyl;
P^{R2} is alkyl;
P^{R3} is each independently hydrogen; and
P^{R4} is alkyl.

“Converted into a prodrug” in the present description means that, as shown in the following reaction formula:

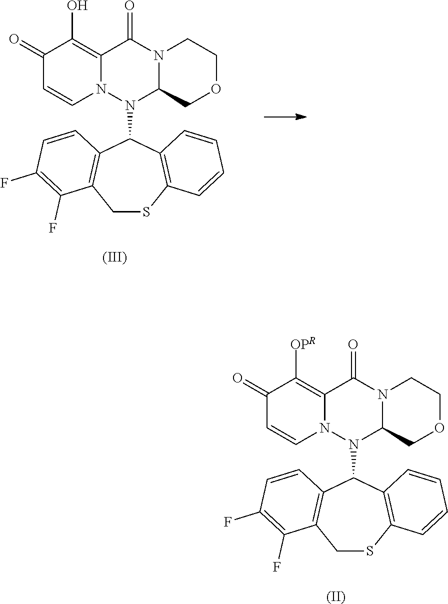

wherein PR is a group to form a prodrug, a hydroxy group in the formula (III) or its pharmaceutically acceptable salt is converted into —OP^{R} group.

“Parent compound” in the present description means a compound to be a source before synthesizing the “prodrug” and/or a compound released from the “prodrug” by the reaction by enzymes, a gastric acid, and the like under physiological conditions in vivo, and specifically means a compound shown by the formula (III), or pharmaceutically acceptable salt thereof or a solvate thereof.

The compounds that are described in PCT application PCT/JP2016/063139 and publication WO 2016/175224A1 are incorporated by reference as examples of one embodiment of the compound in the present description.

The term “alkyl” includes a C1 to C15, alternatively a C1 to C10, alternatively a C1 to C6, alternatively a C1 to C4, linear or branched hydrocarbon group. Examples of “alkyl” include methyl, ethyl, n-propyl, isopropyl, n-butyl, isobutyl, sec-butyl, tert-butyl, n-pentyl, isopentyl, neopentyl, n-hexyl, isohexyl, n-heptyl, isoheptyl, n-octyl, isooctyl, n-nonyl, n-decyl and the like.

One embodiment of “alkyl” is methyl, ethyl, n-propyl, isopropyl, n-butyl, isobutyl, sec-butyl, tert-butyl or n-pentyl. Another embodiment of “alkyl” is methyl, ethyl, n-propyl, isopropyl or tert-butyl.

The term “alkylene” includes a C1 to C15, alternately a C1 to C10, alternately a C1 to C6 and alternately a C1 to C4 linear or branched bivalent hydrocarbon group. Examples include methylene, ethylene, trimethylene, propylene, tetramethylene, pentamethylene, hexamethylene and the like.

One or more hydrogen, carbon and/or other atoms in the compounds used in the present invention may be replaced with isotopes of hydrogen, carbon and/or other atoms respectively. Examples of isotopes include hydrogen, carbon, nitrogen, oxygen, phosphorus, sulfur, fluorine, iodine and chlorine, such as ²H, ³H, ¹¹C, ¹³C, ¹⁴C, ¹⁵N, ¹⁸O, ¹⁷O, ³¹P, ³²P, ³⁵S, ¹⁸F, ¹²³I and ³⁶Cl respectively. The compounds used in the present invention include compounds replaced with these isotopes. The compounds replaced with the above isotopes are useful as medicines and include radiolabeled compounds of the compound used in the present invention. A “method of radiolabeling” in the manufacture of the “radiolabeled compounds” is encompassed by the present invention, and the “radiolabeled compounds” are useful for studies on metabolized drug pharmacokinetics, studies on binding assay and/or diagnostic tools.

A radiolabeled compound used in the present invention can be prepared using well-known methods in the field of this invention. For example, a tritium-labeled compound used in the present invention can be prepared by introducing a tritium to a certain compound used in the present invention, through a catalytic dehalogenation reaction using a tritium. This method comprises reacting with an appropriately-halogenated precursor of the compound used in the present invention with tritium gas in the presence of an appropriate catalyst, such as Pd/C, and in the presence or absent of a base. The other appropriate methods of preparing a tritium-labeled compound can be found in “Isotopes in the Physical and Biomedical Sciences, Vol. 1, Labeled Compounds (Part A), Chapter 6 (1987)”. A ¹⁴C-labeled compound can be prepared by using a raw material having ¹⁴C.

The pharmaceutically acceptable salts of the compounds used in the present invention include, for example, salts with alkaline metal (e.g., lithium, sodium, potassium or the like), alkaline earth metal (e.g., calcium, barium or the like), magnesium, transition metal (e.g., zinc, iron or the like), ammonia, organic bases (e.g., trimethylamine, triethylamine, dicyclohexylamine, ethanolamine, diethanolamine, triethanolamine, meglumine, ethylenediamine, pyridine, picoline, quinoline or the like) or amino acids, or salts with inorganic acids (e.g., hydrochloric acid, sulfuric acid, nitric acid, carbonic acid, hydrobromic acid, phosphoric acid, hydroiodic acid or the like) or organic acids (e.g., formic acid, acetic acid, propionic acid, trifluoroacetic acid, citric acid, lactic acid, tartaric acid, oxalic acid, maleic acid, fumaric acid, mandelic acid, glutaric acid, malic acid, benzoic acid, phthalic acid, ascorbic acid, benzenesulfonic acid, p-toluenesulfonic acid, methanesulfonic acid, ethanesulfonic acid or the like). Especially, salts with hydrochloric acid, sulfuric acid, phosphoric acid, tartaric acid, methanesulfonic acid and the like are included. These salts can be formed by the usual methods.

The compounds used in the present invention or its pharmaceutically acceptable salts may form solvates (e.g., hydrates or the like) and/or crystal polymorphs. The present invention encompasses those various solvates and crystal polymorphs. “Solvates” may be those wherein any numbers of solvent molecules (e.g., water molecules or the like) are coordinated with the compounds used in the present invention. When the compounds used in the present invention or its pharmaceutically acceptable salts are allowed to stand in the atmosphere, the compounds may absorb water, resulting in attachment of adsorbed water or formation of hydrates. Recrystallization of the compounds used in the present invention or its pharmaceutically acceptable salts may produce crystal polymorphs.

The group to form a prodrug is converted into OH group by action of drug-metabolizing enzymes, hydrolases, gastric acids, and/or enterobacteria, after in vivo administration (for example, oral administration).

Additionally, a prodrug shows bioavailability and/or AUC (area under the blood concentration curve) in in vivo administration that is improved compared to that of the compound represented by formula (III).

Therefore, a prodrug is efficiently absorbed into the body in the stomach and/or intestines after in vivo administration (for example, oral administration), and then is converted into the compound represented by formula (III). Thus, the prodrug shows an effect of treating and/or preventing influenza virus infection higher than the compound represented by formula (III).

Examples of one embodiment of the group to form a prodrug include a group selected from the following formulae.

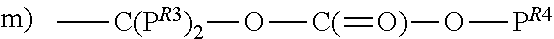

wherein P^{R3} is hydrogen; and P^{R4} is alkyl.

Examples of an embodiment of a particularly preferable substituent of the group to form a prodrug include following groups.

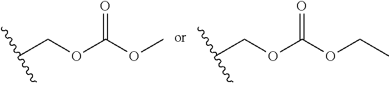

Other compounds that may be used are described in PCT application PCT/JP2016/063139 and publication WO2016/175224A1, all disclosures in which are herein incorporated by reference.

A general method for producing the compound used in the present invention will be exemplified below. As to the extraction and purification, treatment which is performed in a normal experiment of organic chemistry may be conducted.

Synthesis of the compound used in the present invention can be carried out referring to the procedures known in the art.

As a raw material compound, commercially available compounds, compounds described in the present description, compounds described in the references cited in the present description, and other known compounds can be utilized.

When one wants to obtain a salt of the compound used in the present invention, in the case where the compound used in the present invention is obtained in a form of a salt, it may be purified as it is and, in the case where the compound used in the present invention is obtained in a free form, a salt may be formed by a normal method by dissolving or suspending the compound in a suitable organic solvent, and adding an acid or a base.

### (Preparation 1)

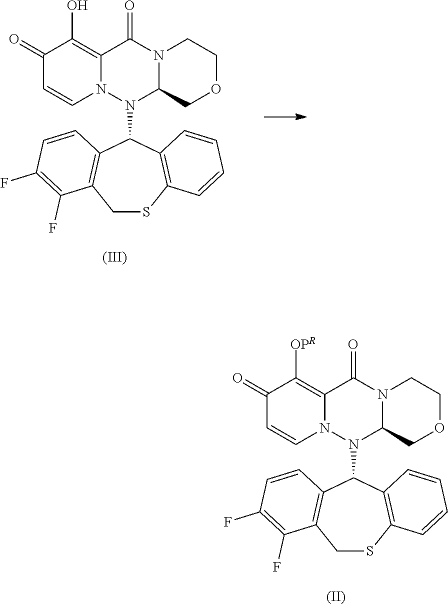

wherein P^{R} is a group to form a prodrug.

Compound (II) can be obtained by the general method including converting a hydroxyl group of Compound (III) into an ester group or ether group. The active agent (Compound (III)) can be used to make its prodrugs (i.e., compounds having the formula of Compound (II)).

For example, the method described in Protective Groups in Organic Synthesis, Theodora W Green (John Wiley & Sons), Prog. Med. 5: 2157-2161 (1985), and Supplied by The British Library—“The World's Knowledge”, etc. can be utilized. These references are herein incorporated by reference.

The parent compound used in the present invention has cap-dependent endonuclease inhibitory activity and the parent compound and its prodrugs are useful as a therapeutic or preventive agent for influenza virus infection.

In general, for the purpose of treating the above-mentioned diseases in humans, the compounds used in the present invention may be administered orally as a powder, a granule, tablets, capsules, pills, a liquid and the like or parenterally as an injection, suppositories, a percutaneous drug, an inhalant and the like. The effective doses of the present compounds may be mixed with excipients suitable for the dosage form, such as fillers, binders, humectants, disintegrators, and lubricants, as appropriate, to form pharmaceutical preparations. For preparing an injection, sterilization is performed with a suitable carrier.

In general, the pharmaceutical compositions used in the present invention can be administered either orally or parenterally. For oral administration, commonly used dosage forms, such as tablets, granule, powder, and capsules, may be prepared according to conventional methods. For parenteral administration, any commonly used dosage form, such as an injection, may be suitably used. The compounds according to the present invention can be suitably used as oral preparations because of their high oral absorbability.

Generally, the dose depends on the condition of the disease, administration route, or age or weight of the patient. The usual oral dose for adults is 0.1 to 100 mg/kg per day, alternately 1 to 20 mg/kg per day. In some embodiments, patients weighing 40 kg to less than 80 kg receive a single dose of 40 mg. In other embodiments, patients weighing at least 80 kg receive a single dose of 80 mg.

In general, the compound used in the present invention can be used in combination with other drugs or the like (hereinafter referred to as combination drugs) to increase the activity of the compound, reduce the dose of the compound, or the like. In the case of treating influenza virus infection, the compound can be used combined with or in a coupled formulation with neuraminidase inhibitor (e.g., Oseltamivir, Zanamivir, Peramivir, Inabiru and the like); RNA-dependent RNA polymerase inhibitor (e.g., Favipiravir); M2 protein inhibitor (e.g., Amantadine); PB2 Cap binding inhibitor (e.g., VX-787); anti-HA antibody (e.g., MHAA4549A); Immune agonists (e.g., Nitazoxanide) are also possible. In this case, the timing of administration for a compound used in the present invention and the combination drug is not limited. They can be administered to the subjects to be treated, at the same time or at different times. Furthermore, a compound used in the present invention and the combination drug can be administered as two or more formulations independently comprising each active ingredient or a single formulation comprising all the active ingredients of the compound of the present inventipon and the combination drug.

The dose for combination drugs may be appropriately selected in reference to the clinical dose. The compounding ratio of the compounds used in the present invention and co-administered drugs may be appropriately selected depending on the subject to be treated, administration route, disease to be treated, symptoms, combination of the drugs and the like. For administration in humans, for example, 1 part by weight of the compounds used in the present invention may be used in combination with 0.01 to 100 parts by weight of co-administered drugs.

In one example, the complication risk factor is one or more of chronic lung diseases, endocrine disorders, being an age that is 65 or older, heart diseases, and morbid obesity.

In one example, the complication risk factor is a chronic lung disease. In one example, the chronic lung disease can include chronic obstructive pulmonary disease (COPD), emphysema, tuberculosis, asthma, interstitial pulmonary disease and cystic fibrosis.

In one example, the complication risk factor is pneumonia. In one example, pneumonia can include aspiration pneumonia.

In one example, the complication risk factor is bronchitis.

In one example, the complication risk factor is an immune disorder due to a disease or medication. In one example, an immune disorder due to a disease or medication can include HIV, AIDS, cancer, T-cell immune deficiency and steroid treatment.

In one example, the complication risk factor is a heart disease. In one example, the heart disease can include congenital heart disease, congestive heart failure and coronary artery disease.

In one example, the complication risk factor is a renal disease. In one example, a renal disease can include chronic renal failure and hemodialysis.

In one example, the complication risk factor is a metabolic disorder. In one example, a metabolic disorder can include inherited metabolic disorders and mitochondrial disorders.

In one example, the complication risk factor is pleural inflammation.

In one example, the complication risk factor is a blood disorder, endocrine disorders, and diabetes. In one example, a blood disorder can include severe anemia including sickle cell disease.

In one example, the complication risk factor is a liver disorder.

In one example, the complication risk factor is being an age younger than 19 years and receiving long-term aspirin therapy.

In one example, the complication risk factor is morbid obesity (for example, body mass index [BMI] of 40 or more).

In one example, the complication risk factor is a neurological and neurodevelopmental condition. In one example, a neurological and neurodevelopmental condition can include cerebral palsy, epilepsy, stroke, intellectual disability, moderate to severe developmental delay, muscular dystrophy, spinal cord injury.

In one example, the complication risk factor is a neuromuscular disorder. In one example, a neuromuscular disorder can include muscular dystrophy, ALS, motor paralysis, spasm, dysphagia, and related neuromuscular diseases.

In one example, the complication risk factor is the age of the subject. In one example, an age factor can include 65 year of age or older.

In one example, the complication risk factor is a race of the subject. In one example, a race factor can include being of Native American heritage, being an Alaskan native, or being an Aboriginal and Torres Strait Islander.

In one example, the complication risk factor can be one or more of the complication risk factors listed above.

In one example, the complication risk factor is one or more of chronic lung diseases, endocrine disorders, being an age that is 65 or older, a current resident of a long-term care facility, metabolic disorders, a compromised immune system, neurological disorders, neurodevelopmental disorders, heart diseases, blood disorders, being a female who is within two weeks postpartum and is not breastfeeding, having American Indian or Alaskan native heritage, and morbid obesity. A more preferred example, the complication risk factor can include one or more of chronic lung diseases, endocrine disorders, being an age that is 65 or older, heart diseases and morbid obesity.

In one example, the amount of the compound administered is effective such that a reduction in a time to improvement of at least one symptom of an influenza virus infection in the subject is statistically significant as compared to that of a non-treated subject. In another example, the amount of the compound administered is effective such that a reduction in a time to improvement of symptoms of an influenza virus infection (coughing, sore throat, headache, nasal congestion, feverishness or chills, muscular or joint pain, and fatigue) in the subject is statistically significant as compared to that of a non-treated subject. In one example, a non-treated subject is a subject that has been administered the placebo of the compound or a subject that has not been administered the compound.

In one example, the time to improvement of at least one symptom in the subject where the compound is administered is a time from the first administration of the compound to an improvement of influenza symptoms, as compared to the respective symptoms before the administration of the compound, for at least 24 hours.

In one example, the time to improvement of at least one symptom in the subject where the placebo is administered or the compound is not administered is a time from the initial administration of the placebo; or if the compound is not administered the corresponding time point in view of the disease process of influenza to an improvement of influenza symptoms, as compared to the respective symptoms before the administration of the placebo; or if the compound is not administered the corresponding time point in view of the disease process of influenza.

In one example, the time to improvement of at least one symptom in the subject is a time from the initial administration of the compound to an improvement of influenza symptoms, as compared to the respective symptoms before the administration of the compound, for at least 48 hours.

In one example, the time to improvement of at least one symptom in the subject is a time from the initial administration of the compound to an improvement of influenza symptoms, as compared to the respective symptoms before the administration of the compound, for at least 72 hours.

In one example, the time to improvement of at least one symptom in the subject is a time from the initial administration of the compound to an improvement of influenza symptoms, as compared to the respective symptoms before the administration of the compound, for at most 86 hours.

In one example, a reduction in the time to improvement of at least one symptom of the influenza infection is statistically significant relative to that of a non-treated subject, where a p-value indicating the statistical significance is less than 0.05, alternately 0.03 or less, alternately 0.02 or less, alternately 0.003 or less, alternately 0.001 or less, alternately 0.001 or less.

In one example, a symptom of an influenza virus infection in a subject is a systemic symptom or a respiratory symptom. In one example, a systemic symptom includes one or more of headache, feverishness, chills, muscular pain, joint pain, and fatigue. In one example, a respiratory symptom includes one or more of coughing, sore throat, and nasal congestion.

The phrase “improvement of a symptom of an influenza virus infection” refers to a self-evaluation of the subject's influenza symptoms using a 4-point scale [0: none, 1: mild, 2: moderate, 3: severe] starting from the time the compound or the placebo is initially administered; or if the compound is not administered from the corresponding time point in view of the disease process of influenza. Seven influenza symptoms are evaluated, which are cough, sore throat, headache, nasal congestion, feverishness or chills, muscular or joint pain, and fatigue. Improvement occurs when all seven influenza symptoms (cough, sore throat, headache, nasal congestion, feverishness or chills, muscular or joint pain, fatigue) become lower relative to the time the compound or the placebo is initially administered; or if the compound is not administered to the corresponding time point in view of the disease process of influenza. Alternatively, improvement of any particular influenza symptom refers to when the influenza symptom returns to the patient's baseline level; a self-evaluation point reduces by at least by 1 level if pre-existing symptoms are worsened at baseline by influenza; and/or a self-evaluation point is not changed if pre-existing symptoms are not worsened at baseline by influenza.

In one example, the subject has a type B influenza virus. In one example, the time to improvement of symptoms is statistically significant relative to that of a subject that has been administered oseltamivir.

In one example, a p-value indicating the statistical significance of the time to improvement of at least one symptom of the influenza virus infection where the subject has a type B influenza virus is less than 0.05, alternately 0.03 or less, alternately 0.02 or less, alternately 0.003 or less, alternately 0.001 or less, alternately 0.001 or less.

In one example, the amount of the compound administered is effective such that an avoidance and/or a reduction in an incidence of an influenza-related complication in the subject is statistically significant as compared to that of a non-treated subject.

In one example, a p-value indicating the statistical significance of the reduction in an incidence of an influenza-related complication is less than 0.05, alternately 0.03 or less, alternately 0.02 or less, alternately 0.003 or less, alternately 0.001 or less, alternately 0.001 or less.

In one example, the influenza-related complication is death. In one example, the influenza-related complication is hospitalization. In one example, the influenza-related complication is sinusitis. In one example, the influenza-related complication is otitis media. In one example, the influenza-related complication is bronchitis. In one example, the influenza-related complication is pneumonia. In one example, the influenza-related complication is one or more of those listed above. In one example, the influenza-related complication is one or more of the group consisting of sinusitis and bronchitis.

In one example, the amount of the compound administered is effective such that a reduction in a time to cessation of viral shedding by a virus titer in the subject is statistically significant as compared to that of a non-treated subject. In one example, the time to cessation of viral shedding by a virus titer means a time between the initial administration of the compound or the placebo to the subject having an influenza virus infection; or if the compound is not administered the corresponding time point in view of the disease process of influenza and the first time when the virus titer or viral ribonucleic acid (RNA) of the subject as measured by reverse transcription polymerase chain reaction (RT-PCR) is less than the lower limit of quantification. In some examples, the lower limit of quantification is a baseline at certain timepoints. In some examples, the time to cessation of viral shedding by a virus titer means a time between the initial administration of the compound or the placebo to the subject having at least one symptom of an influenza virus infection; or if the compound is not administered the corresponding time point in view of the disease process of influenza and the time when no viral shedding from the subject is detected for the first time after the initial administration of the compound, with the virus titer or viral RNA virus titer or viral ribonucleic acid (RNA) of the subject as measured by reverse transcription polymerase chain reaction (RT-PCR).

In one example, a p-value indicating the statistical significance of the reduction in a time to cessation of viral shedding by a virus titer in the subject is less than 0.05, alternately 0.03 or less, alternately 0.02 or less, alternately 0.003 or less, alternately 0.001 or less, alternately 0.001 or less.

In one example, the amount of the compound administered amount is effective such that a reduction of a virus titer in the subject is statistically significant as compared to that of a non-treated subject. In one example, the virus titer in the subject is reduced by at least about 2.8 log₁₀ TCID₅₀/mL, alternately at least about 3.3 log₁₀ TCID₅₀/mL, relative to that of when the compound is first administered to the subject. In one example, the reduction in the virus titer is measured on Day 2 after the compound is first administered to the subject. “Day 2” means one day after the compound is first administered to the subject.

In one example, a subject that is first administered with the compound has a virus titer sufficient to cause a symptom of an influenza virus infection to be exhibited in the subject. In one example, the virus titer sufficient to cause a symptom of influenza virus infection to be exhibited in the subject is 0.7 log₁₀ TCID₅₀/mL.

In one example, a p-value indicating the statistical significance of the reduction of a virus titer in the subject is less than 0.05, alternately 0.03 or less, alternately 0.02 or less, alternately 0.003 or less, alternately 0.001 or less, alternately 0.001 or less.

In one example, the effective amount of the compound is in a range from about 0.1 mg to about 3000 mg. In another example, the effective amount of the compound is in a range from about 0.1 to about 240 mg. In another example, the effective amount of the compound is in a range from about 3 mg to about 80 mg. In yet another example, the effective amount of the compound is in a range from about 40 mg to about 80 mg. In yet another example, the effective amount is in a range from about 3 mg to about 80 mg per dose.

In one example, the subject is a human patient.

In one example, the compound is administered based on the weight of the subject. In one example, the compound can be administered as a weight-based dose. In one example, about 40 mg is administered to a subject weighing from about 40 kg to under about 80 kg. In one example, about 80 mg is administered to a subject weighing 80 kg or more. In one example, the compound is administered on the first day of onset of at least one symptom of an influenza virus infection and three days after the first day of administration upon onset of at least one symptom of an influenza virus infection. In one example, the compound is administered once.

In one example, the compound is administered six days after the first day of administration if improvement has not occurred four days after the first day of administration. In some examples, improvement means a lower score in seven of the influenza symptoms (cough, sore throat, headache, nasal congestion, feverishness or chills, muscular or joint pain, and fatigue) using a 4-point scale [0: none, 1: mild, 2: moderate, 3: severe] relative to the time the compound is initially administered. Alternatively, improvement of any particular influenza symptom refers to returning of the influenza symptom to the patient's baseline level.

In some examples, improvement means: a reduction in a self-evaluation point at least by one level if pre-existing symptoms are worsened by influenza compared with a baseline; a self-evaluation point is not changed if pre-existing symptoms are not worsened by influenza compared with a baseline; and a self-evaluation point becomes mild or absent if symptoms are not pre-existing.

In one example, the compound is administered orally. In another example, the compound is administered parenterally.

In one example, the compound is administered through at least one route selected from the group consisting of orally, dermally, subcutaneously, intravenously, intraarterially, intramuscularly, intraperitoneally, transmucosally, via inhalation, transnasally, ophthalmically, via an inner ear and vaginally.

Generally, the compound can be administered with any material in any amounts that are suitable for use with the compound. In one example, the compound is administered in combination with at least one material selected from the group consisting of a neuraminidase inhibitor, an RNA-dependent RNA polymerase inhibitor, an M2 protein inhibitor, a PB2 Cap binding inhibitor, a HA maturation inhibitor, a recombinant sialidase, a re-assemble inhibitor, RNA interference compound, a receptor of hemagglutinin binding inhibitor, a membrane of HA fusion inhibitor, a NP nuclear translocation inhibitor, a CXCR inhibitor, a CRM1 inhibitor, an anti-HA antibody and an immunological agent.

In one example, the compound is administered in combination with one or more of oseltamivir, zanamivir, peramivir, laninamivir, favipiravir, amantazine, flumazine,

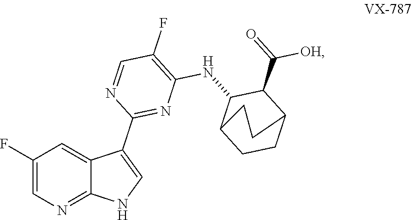

MHAA4549A (as described in McBride et al., Antimicrobial Agents and Chemistry, Vol. 61, Issue 11, (2017)), TCN-032 (as described in Ramos et al., JID 2015:11 (2015)), VIS-410 (as described in Tharakaraman et al., PNAS, vol. 112, no. 35, 10890-10895 (2015)), CR-8020 (as described in Ekiert et al., Science, 333(6044), 843-850 (2011)), CR-6261 (as described in Ekiert et al., Science, 324(5924), 246-251 (2009)), CT-P27 (as described in Celltrion, Press Release, Oct. 12, 2016) and MEDI-8852 (as described in Cell, 166(3), 596-608 (2016)).

In one example, the compound is administered in at least one form selected from the group consisting of a tablet, powder, a granule, a capsule, a pill, a film, a suspension, an emulsion, an elixir, a syrup, lemonade, spirit, aromatic water, extract, decoction and tincture. In one example, the compound is administered in a tablet.

In one example, the compound is administered in at least one form selected from the group consisting of a sugar-coated tablet, a film-coated tablet, an enteric-coated tablet, a sustained-release tablet, a troche tablet, a sublingual tablet, a buccal tablet, a chewable tablet, an orally disintegrated tablet, a dry syrup, a soft capsule, a micro capsule or a sustained-release capsule.

In one example, the compound is administered in at least one form selected from the group consisting of an injection, an infusion, an eye drop, a nose drop, an ear drop, an aerosol, an inhalation, a lotion, an impregnation, a liniment, a mouthwash, an enema, an ointment, a plaster, a jelly, a cream, a patch, a cataplasm, an external powder or a suppository.

### EXAMPLES

The present invention will be explained in more detail below by way of Examples, as well as Test Examples of the present invention, but the present invention is not limited to them.

The NMR analysis obtained in each reference example and example was carried out in 300 MHz, and was measured using DMSO-d₆, CDCl₃.

The term RT represents a retention time at LC/MS: liquid chromatography/mass spectrometry, and was measured under the following conditions.

### Measurement Conditions

(1) Column: ACQUITY UPLC (Registered trademark) BEH C18 (1.7 μm i.d.2.1×50 mm)

### Waters

Flow rate: 0.8 mL/min
UV detection wavelength: 254 nm
Mobile phase: [A]: a 0.1% formic acid-containing aqueous solution, [B]: a 0.1% formic acid-containing acetonitrile solution
Gradient: a linear gradient of 5% to 100% solvent [B] was carried out in 3.5 minutes, and 100% solvent [B] was kept for 0.5 minutes.

### Example 1

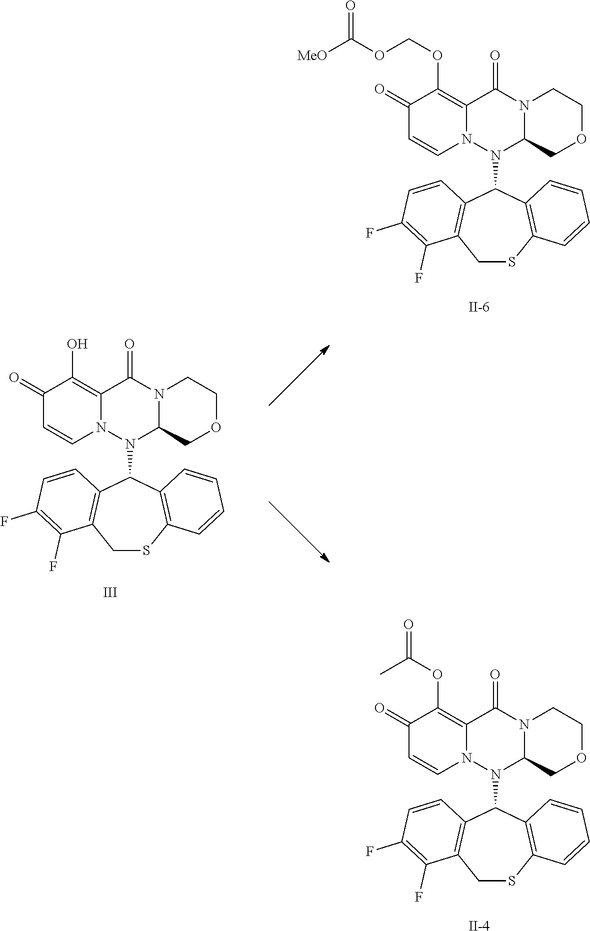

Compound II-4 and II-6 were synthesized from commercially available compounds according to the method described in WO2016/175224.

### Compound II-6

1H-NMR (DMSO-D6) δ: 2.91-2.98 (1H, m), 3.24-3.31 (1H, m), 3.44 (1H, t, J =10.4 Hz), 3.69 (1H, dd, J =11.5, 2.8 Hz), 3.73 (3H, s), 4.00 (1H, dd, J =10.8, 2.9 Hz), 4.06 (1H, d, J =14.3 Hz), 4.40 (1H, d, J =11.8 Hz), 4.45 (1H, dd, J =9.9, 2.9 Hz), 5.42 (1H, dd, J =14.4, 1.8 Hz), 5.67 (1H, d, J =6.5 Hz), 5.72-5.75 (3H, m), 6.83-6.87 (1H, m), 7.01 (1H, d, J =6.9 Hz), 7.09 (1H, dd, J =8.0, 1.1 Hz), 7.14-7.18 (1H, m), 7.23 (1H, d, J =7.8 Hz), 7.37-7.44 (2H, m).

### Compound II-4

1H-NMR(CDC13)δ:2.46(s, 3H), 2.88-2.99(m, 1H), 3.35-3.50(m, 1H), 3.60-3.65(m, 1H), 3.75-3.83(m, 1H), 3.90-4.00(m, 1H), 4.05(d, J=14.0Hz, 1H), 4.52-4.57(m, 1H), 4.60-4.70(m, 1H), 5.24-5.34(m, 1H), 5.35(s, 1H), 5.88(d, J=7.6Hz, 1H), 6.85-6.82(m, 1H), 6.90-7.05(m, 2H), 7.06-7.20(m, 4H)
LC/MS (ESI):m/z =526.2 [M+H]⁺, RT=1.87 min, method (1)

The following example compounds in Table 1 were synthesized from commercially available compounds according to the above examples and reference.

TABLE 1

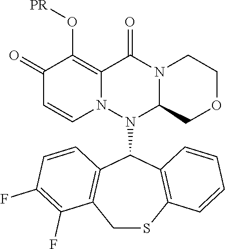

| No. | PR | data |
|---|---|---|
| II-5 | 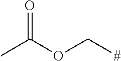 | 1H-NMR(DMSO-d6) δ: 2.04(s, 3H), 2.90-3.00(m, 1H), 3.44-3.50(m, 2H), 3.64-3.72(m, 1H), 3.95-4.00(m, 1H), 4.11-4.10(m, 1H), 4.20- 4.30(m, 2H), 5.40-5.5.46(m, 1H), 6.62-5.75(m, 4H), 6.80-6.90(m, 1H), 6.98-7.10(m, 1H), 7.11-7.20(m, 2H), 7.21-7.30(m, 1H), |
| | | 7.45-7.50(m, 2H) |
| II-7 | 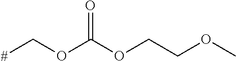 | 1H-NMR(CDCl3) δ: 2.85-2.97 (m, 1H), 3.38 (s, 3H), 3.39-3.48 (m, 1H), 3.54 (t, J = 10.4 Hz, 1H), 3.68 (t, J = 4.4 Hz, 2H), 3.74 (dd, J = 2.8 Hz, 12.0 Hz, 1H), 3.92 (dd, J = 2.8 Hz, 10.8 Hz, 1H), 4.05 (d, J = 13.6 Hz, 1H), 4.36 (q, J = 4.4 Hz, 2H), 4.51 (dd, J = 2.8 Hz, 9.6 |
| | | Hz, 1H), 4.65 (d, J = 12.0 Hz, 1H), 5.27 (dd, J = 2.0 Hz, 13.6 Hz, |
| | | 1H), 5.34 (s, 1H), 5.86 (d, J = 8.0 Hz, 1H), 5.93 (s, 2H), 6.81-6.89 |
| | | (m, 2H), 6.98-7.15 (m, 5H). |
| II-8 | 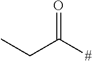 | 1H-NMR (CDCl3) δ: 1.33 (3H, t, J = 7.0 Hz), 2.82 (2H, d, J = 6.1 Hz), 2.93 (1H, t, J = 11.2 Hz), 3.42 (1H, t, J = 11.4 Hz), 3.59 (1H, t, J = 10.2 Hz), 3.78 (1H, d, J = 11.2 Hz), 3.96 (1H, d, J = 10.3 Hz), 4.06 (1H, d, J = 13.8 Hz), 4.55 (1H, d, J = 8.9 Hz), 4.63 (1H, d, J = |
| | | 13.6 Hz), 5.29 (1H, d, J = 13.9 Hz), 5.36 (1H, s), 5.88 (1H, d, J = |
| | | 7.4 Hz), 6.90 (1H, s), 7.03-7.12 (6H, m). |
| II-9 | 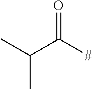 | 1H-NMR (CDCl3) δ: 1.42 (d, J = 6.8 Hz, 6H), 2.85-3.05 (m, 2H), 3.40-3.49 (m, 1H), 3.59 (t, J = 10.4 Hz, 1H), 3.76 (d, J = 11.4 Hz, 1H), 3.94 (d, J = 10.4 Hz, 1H), 4.06 (d, J = 14.1 Hz, 1H), 4.51-4.57 (m, 1H), 4.59-4.70 (m, 1H), 5.25-5.32 (m, 1H), 5.35-5.39 (m, 1H), 5.80-5.89 (m, 1H), 6.85-7.15 (m, 7H). |
| II-10 | 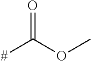 | LC/MS (ESI): m/z = 542 [M + H]+, RT = 1.92 min, method (1) |
| II-11 | 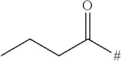 | LC/MS (ESI): m/z = 554 [M + H]+, RT = 2.10 min, method (1) |

### Test Example 1: BA test

### Materials and Methods for Experiments to Evaluate Oral Absorption

(1) Experimental animals: mice or SD rats were used.
(2) Rearing condition: mice or SD rats were fasted and were allowed free access to sterilized tap water.
(3) Setting of dosage and grouping: Oral administration and intravenous administration were performed with the predetermined dosage. Grouping was set as below. (Dosage was changed per compound)
   Oral administration 1 to 30 mg/kg (n=2 to 3)
   Intravenous administration 0.5 to 10 mg/kg (n=2 to 3)
(4) Preparation of administration solutions: Compounds II-4, II-5, II-6, II-7, II-8, II-9, II-10, and II-11 were prepared for evaluation in rats. Oral administration was performed as solution or suspension. Intravenous administration was performed after solubilization.
(5) Routes of administration: Oral administration was performed mandatory into the stomach by oral sonde. Intravenous administration was performed from caudal vein by syringes with needle.
(6) Evaluation items: Blood was collected serially and concentration of a compound used in the present invention in plasma was measured by LC/MS/MS.
(7) Statistical analysis: About transition of concentration of a compound used in the present invention in plasma, the area under the plasma concentration versus time curve (AUC) was calculated by non-linear least-squares method program, WinNonlin (a registered trademark), and bioavailability (BA) of a compound used in the present invention was calculated from AUCs of the oral administration group and the intravenous administration group. The BAs of each compound are described in Table 2 below.

### Result

TABLE 2

| No. | BA(%) |
|---|---|
| II-4 | 20.0 |
| II-5 | 17.8 |
| II-6 | 14.9 |
| II-7 | 14.5 |
| II-8 | 27.8 |
| II-9 | 15.0 |
| II-10 | 10.6 |
| II-11 | 11.0 |
| III | 4.2 |

Based on the above results, all of the prodrug compounds had improved bioavailability compared to Compound III.

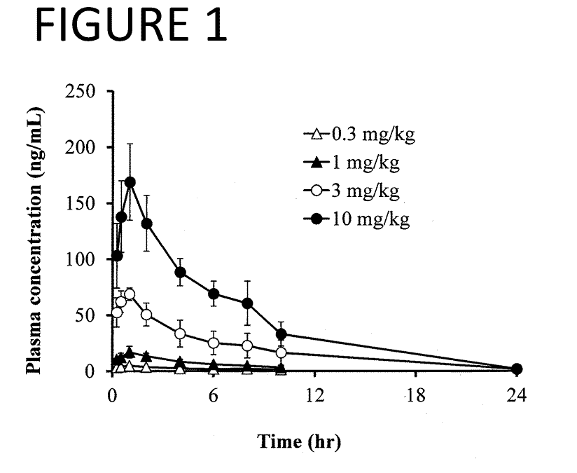

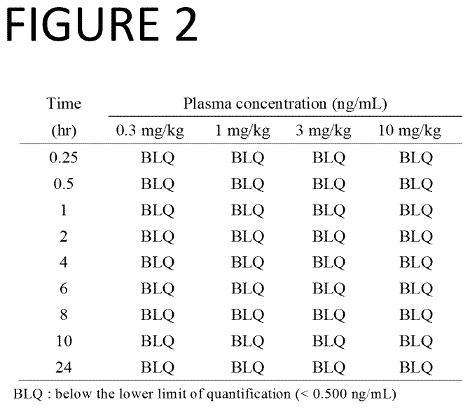

FIGS. 1 and 2 show a result of measuring the plasma concentration of Compound III and Compound II-6, respectively, after oral administration of prodrug Compound II-6 to rats under non-fasting conditions.

As shown in FIG. 2, the concentration of Compound II-6 in all plasma samples was below the limit of quantification (<0.500 ng/mL) for all time points tested (0.25 h, 0.5 h, 1 h, 2 h, 4 h, 6 h, 8 h, 10 h, and 24 h) and for all doses tested (0.3 mg/kg, 1 mg/kg, 3 mg/kg, and 10 mg/kg). Therefore, prodrug Compound II-6 was found to have metabolized promptly to Compound III in vivo after administration.

Based on the above test results, it was revealed that the prodrug compunds were absorbed into the body after oral administration, and rapidly converted into Compound III in the blood. The prodrug compounds used in the present example also showed excellent oral absorbability. Therefore, the prodrug compounds used in the present example, including Compound II-6, can be useful agents for treatment and/or prevention of symptom and/or disease induced by infection with influenza virus.

### Test Example 2: Clinical Trial

The efficacy and safety of a single oral administration of BXM (40 mg or 80 mg) was evaluated in patients who were infected with influenza virus and were symptomatic for no more than 48 hours, and had a complication risk factor. The patients were evaluated by a randomized, double-blind, multicenter, placebo- and active-controlled global study designed to evaluate the efficacy and safety of a single oral dose of BXM compared with placebo or oseltamivir, in adult and adolescent subjects aged 12 to 17 years with both influenza virus infection and a complication risk factor.

A total of 2,184 high risk subjects were randomized to receive a single oral dose of 40 mg or 80 mg of BXM according to body weight (patients who weighed from 40 kg to less than 80 kg received 40 mg and patients who weighed more than or equal to 80 kg received 80 mg), oseltamivir 75 mg twice daily for 5 days, or placebo. The predominant influenza viruses in this study were the subtype A/H3N2 (47.9%) and type B (41.6%). The primary efficacy endpoint was a time to improvement of influenza symptoms (cough, sore throat, headache, nasal congestion, feverishness or chills, muscle or joint pain, and fatigue).
(1) Patients who satisfied all of the following criteria were selected as subjects.
(1.1) Male or female patients at 12 years old or older,
(1.2) Patients with a diagnosis of influenza virus infection confirmed by all of the followings:
a) Fever ≥38° C. (axillary) in the predose examinations or >4 hours after dosing of antipyretics if they were taken,
b) A positive rapid influenza diagnostic test (RIDT) result or a patient with a negative RIDT may be enrolled if the patient reports contact with a person known to have influenza virus infection within 7 days prior to the treatment and if all other inclusion criteria are met,
c) At least one each of the following general and respiratory symptoms associated with influenza virus infection with a severity of moderate or greater is present:
i) General symptoms (headache, feverishness or chills, muscle or joint pain, or fatigue)
ii) Respiratory symptoms (cough, sore throat, or nasal congestion)
(1.3) The time interval between the onset of symptoms and the predose examinations is 48 hours or less, where the onset of the symptoms is defined as either
a) Time of the first increase in body temperature (as an increase of at least 1° C. from the patient's normal body temperature), or
b) Time when the patient experiences at least one new general or respiratory symptom above,
(1.4) If a women having childbearing potential is the patient, she agrees to use a highly effective method of contraception for 3 months after the first dose of study drug,
(1.5) Patients are considered as having a complication risk factor due to the presence of at least one of the following inclusion criteria:
a) Asthma or chronic lung disease (such as chronic obstructive pulmonary disease or cystic fibrosis),
b) Endocrine disorders (including diabetes mellitus),
c) Residents of long-term care facilities (e.g., nursing homes),
d) Compromised immune system (including patients receiving corticosteroids not exceeding 20 mg of prednisolone or equivalent, and patients being treated for human immunodeficiency virus [HIV] infection with a CD4 count >350 cells/mm³ within the last 6 months),
e) Neurological and neurodevelopmental disorders (including disorders of the brain, spinal cord, peripheral nerve, and muscle, e.g., cerebral palsy, epilepsy [seizure disorders], stroke, muscular dystrophy, or spinal cord injury),
f) Heart diseases (such as congenital heart disease, congestive heart failure, or coronary artery disease), excluding hypertension without any other heart-related symptoms,
g) Adults aged ≥65 years,
h) American Indians and Alaskan Natives,
i) Blood disorders (such as sickle cell disease),
j) Metabolic disorders (such as inherited metabolic disorders and mitochondrial disorders),
k) Morbid obesity (body mass index ≥40 kg/m²), and
l) Women who are within 2 weeks postpartum and are not breastfeeding.
(2) Method for administering investigational drug
(i) Test drug
20 mg Tablet of BXM
(ii) Placebo or control drug
Placebo for 20 mg tablet of BXM
75 mg Capsule of Oseltamivir
Placebo for 75 mg capsule of Oseltamivir
(3) Dosage and administration method

Eligible patients were randomly allocated to a group receiving a single administration of BXM (40 or 80 mg depending on the body weight), a group receiving 75 mg Oseltamivir twice a day for 5 days, and a placebo group in a ratio of 1:1:1.

The dosage of BXM was 40 mg for subjects weighing less than 80 kg, and 80 mg for subjects weighing 80 kg or more.
(4) Investigational drug for each administered group

As used below, the term “Day 1” indicates the first day of administration. The term “Day 2 to Day 5” indicates the second day to the fifth day as counted from the first day of administration.

### [BXM Group]

### Day 1:

Single dose of 40 mg Tablets of BXM was administered orally to patients who weighed between 40 kg and up to 80 kg (Two 20 mg tablets). Single dose of 80 mg Tablets of BXM was administered orally to patients who weighed greater than or equal to 80 kg (Four 20 mg tablets). Placebo capsules for Oseltamivir were administered orally twice a day (morning, evening), one capsule per administration.

### Day 2 to Day 5:

Placebo capsules for Oseltamivir were administered orally twice a day (morning, evening), one capsule per administration.

### [Oseltamivir Group]

### Day 1:

Placebo tablets for BXM were administered orally. 75 mg Capsules of Oseltamivir were administered orally twice a day (morning, evening), one capsule per administration.

### Day 2 to Day 5:

75 mg Capsules of Oseltamivir were administered orally twice a day (morning, evening), one capsule per administration.

### [Placebo Group]

### Day 1:

Placebo tablets for BXM were administered orally (2 tablets or 4 tablets depending on the body weight). Placebo capsules for Oseltamivir were administered orally twice a day (morning, evening), one capsule per administration.

### Day 2 to Day 5:

Placebo capsules for Oseltamivir were administered orally twice a day (morning, evening), one capsule per administration.
(5) Main efficacy endpoint

The main efficacy endpoint is the time to alleviation of influenza symptoms, which is the time from the beginning of administration until improvement of influenza symptoms for at least 21.5 hours. Improvement of influenza symptoms refers to when all 7 influenza symptoms (cough, sore throat, headache, nasal congestion, feverishness or chills, muscular or joint pain, and fatigue) become “0: none” or “1: mild” in the patient diary that the subject keeps, and this condition continues at least 21.5 hours (24 hours—10%). Alternatively, improvement of any particular influenza symptom refers to when the influenza symptom returns to the patient's baseline level.
(5.1) Preexisting symptoms (i.e., cough, fatigue, or muscle/joint pain that existed prior to developing influenza virus infection) that were judged by the patient to be worse at baseline (i.e., the predose examinations) must improve from baseline severity.
(i) Improvement of severity as compared to the baseline severity is as follows:
(a) Severity changed from severe to moderate, mild, or none, or
(b) Severity changed from moderate to mild or none

The baseline severity is the severity of the symptoms immediately before administering the compound to the patient. The baseline severity is assessed as severe, moderate, mild or none. If the baseline severity is severe, then it is necessary to administer the compound so that the baseline severity is moderate, mild, or none.

At baseline (i.e., the predose examinations), patients only is asked whether preexisting symptoms existed (within the last 30 days) and whether they were worsened by influenza virus infection. Patients are asked to rate the severity at baseline that is the severity that needs to improve. To avoid recall bias, patients will not be asked to rate the severity of preexisting symptoms prior to influenza.
(5.2) Preexisting symptoms (i.e., cough, fatigue, or muscle/joint pain that existed prior to developing influenza) that were judged by the patient not to be worse than the baseline (i.e., the predose examinations) must have their baseline severity maintained. Maintaining the baseline severity means that the baseline severity is neither worsened nor improved.
(i) Maintenance of baseline severity is as follows:
No change of baseline severity from severe after administration of the compound
No change of baseline severity from moderate after administration of the compound
(6) Secondary efficacy endpoint

In one example, at least one of the efficacy endpoints is satisfied.

The secondary efficacy endpoint is as follows:
(6.1) Change from baseline in virus titer and in the amount of virus (RT-PCR) at each time point
(6.2) Time to cessation of viral shedding by virus titer and by RT-PCR
(6.3) Time to alleviation of symptoms (cough, sore throat, headache, nasal congestion, feverishness or chills, muscle or joint pain, and fatigue)
(6.4) Incidence of influenza-related complications (hospitalization, death, sinusitis, bronchitis, otitis media, and radiologically confirmed pneumonia)
(7) The virus titer was measured in the following manner:
(7.1) MDCK-SIAT1 cells seeded in a flat-bottom 96-well microplate are cultured in a 5% CO2 incubator at 37±1° C. for 1 day (7.2) A standard strain (influenza virus AH3N2, A/Victoria/361/2011, storage condition: −80° C., origin: National Institute of Infectious Diseases), a sample (collected from high risk patients in Phase III clinical test of BXM and stored in an ultra-low-temperature freezer), and a medium for cell control are diluted 101 to 107 folds by a 10-fold serial dilution method.
(7.3) After cells present in a sheet form are confirmed under an inverted microscope, the medium was removed, and a new medium is added at 100 L/well.
(7.4) The medium is removed.
(7.5) Each of the samples (100 to 107) prepared in (2) above is inoculated at 100 μL/well, using 4 wells per sample.
(7.6) Centrifugal adsorption is performed at room temperature at 1000 rpm for 30 minutes.
(7.7) After centrifugation, the medium is removed, and cells were washed once with a new medium.
(7.8) A new medium is added at 100 μL/well.
(7.9) Incubation is performed in a 5% CO2 incubator at 33±1° C. for 3 days.
(7.10) After incubation, the CytoPathic Effect (CPE) is evaluated under an inverted microscope.
(8) Statistical Methods:

The intention-to-treat infected (ITTI, defined as RT-PCR positive for influenza) set was the primary efficacy analysis population in the study. The per-protocol set (PPS) was used to support the primary analyses for efficacy. Statistical testing was performed at the 2-sided significance level of 0.05 unless stated otherwise.
(9) Analysis of primary endpoint
(9.1) Primary Analysis

The stratified generalized Wilcoxon test was applied to the primary endpoint with some stratification factors, namely baseline symptom score (≤14, ≥15), preexisting and worsened symptom (Yes, No), and region (Asia, North America/Europe, Southern Hemisphere), to evaluate the efficacy of BXM compared with placebo.
(9.2) Secondary Analysis

The same analysis method and endpoint as the primary analysis were used to evaluate the efficacy of BXM compared with oseltamivir.

Together with the primary efficacy analysis, this comparison was conducted in a hierarchical manner so as to maintain control of overall type I error. For Japan, control of overall type I error was not required for the secondary efficacy analysis of primary endpoint.

The same analysis in the PPS was performed as a sensitivity analysis.
(9.3) Other Analyses

In addition, a Kaplan-Meier survival curve was plotted for each group, and the median times, the differences of the median times, and their 95% CIs was calculated.

The same analysis in PPS was performed as a sensitivity analysis.
(10) Analyses of Secondary Endpoints
(10.1) Change from the baseline in virus titer and the amount of virus RNA (RT-PCR) at each time point

Only patients whose virus titer/RT-PCR predose at Visit 1 were ≥ the lower limit of quantification were included in the analyses. The van Elteren test was used at each time point to compare BXM with oseltamivir/placebo, where baseline symptom score (≤14, ≥15), preexisting and worsened symptom (Yes,No), and region (Asia, North America/Europe, Southern Hemisphere) were included as stratification factors. Summary statistics were calculated by a time point and by a treatment group.
(10.2) Time to cessation of viral shedding by virus titer and by RT-PCR

Only patients whose virus titer/RT-PCR predose at Visit 1 were ≥ the lower limit of quantification were included in the analyses. The same analyses as the primary endpoint were performed.
(10.3) Time to alleviation of symptoms

The same analyses as the primary endpoint were performed.
(10.4) Incidence of influenza-related complications (hospitalization, death, sinusitis, bronchitis, otitis media, and radiologically-confirmed pneumonia)

A summary table was created. Fisher's exact test was used to compare the incidence between BXM and oseltamivir/placebo.

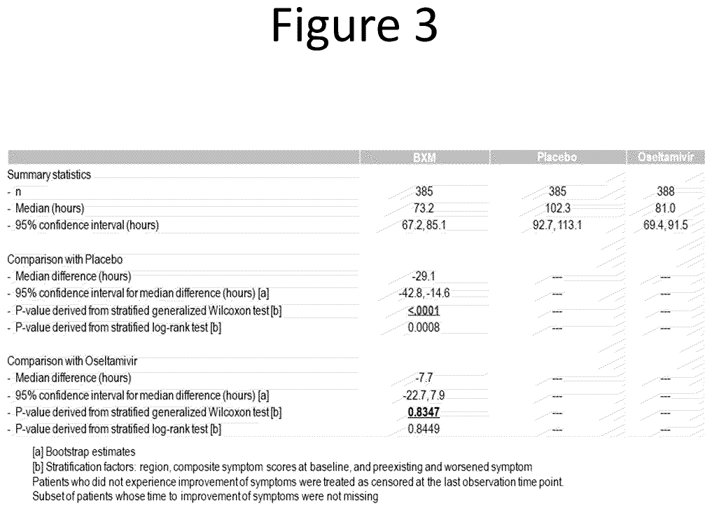

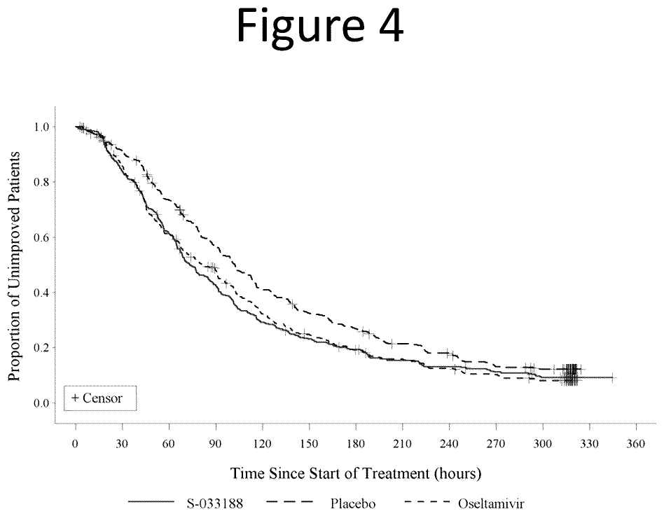

A statistically significant improvement in the primary endpoint was observed for BXM when compared with placebo (see summary of results in Table 3 below). Details of the results are provided in the table in FIG. 3 and the graph in FIG. 4.

TABLE 3
Time to Improvement of Influenza Symptoms (BXM vs Placebo)

| BXM 40/80 mg | Placebo | |
|---|---|---|
| (95% CI¹) | (95% CI¹) | Difference between BXM and placebo² |
| N = 385 | N = 385 | (95% CI for difference) |
| 73.2 | 102.3 | −29.1 |
| (67.2, 85.1) | (92.7, 113.1) | (−42.8, −14.6) |

¹CI: Confidence Interval
²BXM treatment resulted a significant reduction in Time to Improvement of Influenza Symptoms compared to placebo controlled using Peto-Prentice's generalized Wilcoxon test (p-value: <0.0001)

As for the primary endpoint, subjects made evaluations by themselves on a 4-point scale [0: none, 1: mild, 2: moderate, 3: severe] concerning the time to improvement of influenza symptoms for at least 21.5 hours (the time from the beginning of administration of the investigational drug until all seven influenza symptoms (“cough”, “sore throat”, “headache”, “nasal congestion”, “feverishness or chills”, “muscular or joint pain”, and “fatigue”) were improved for at least 21.5 hours) to evaluate the efficacy of the investigational drug over the placebo. The primary efficacy endpoint is a time to improvement of influenza symptoms (TTIIS), which is defined as the time from the start of treatment to the time when all seven influenza-related symptoms were rated by the patients as improved (reduced by at least by 1 level if pre-existing symptoms were worsened at baseline by influenza, not changed if pre-existing symptoms were not worsened at baseline by influenza, or mild or absent if symptoms were not pre-existing).

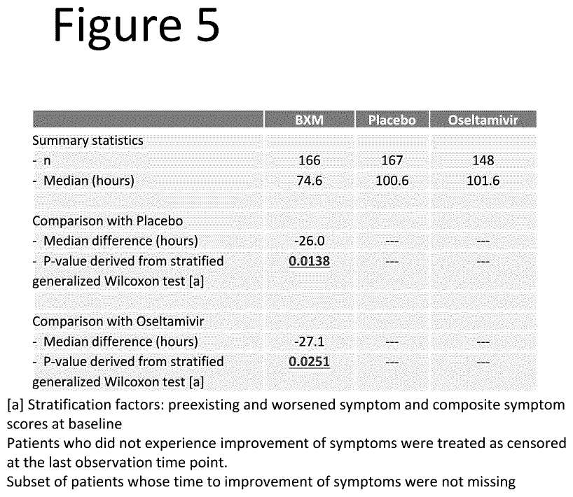

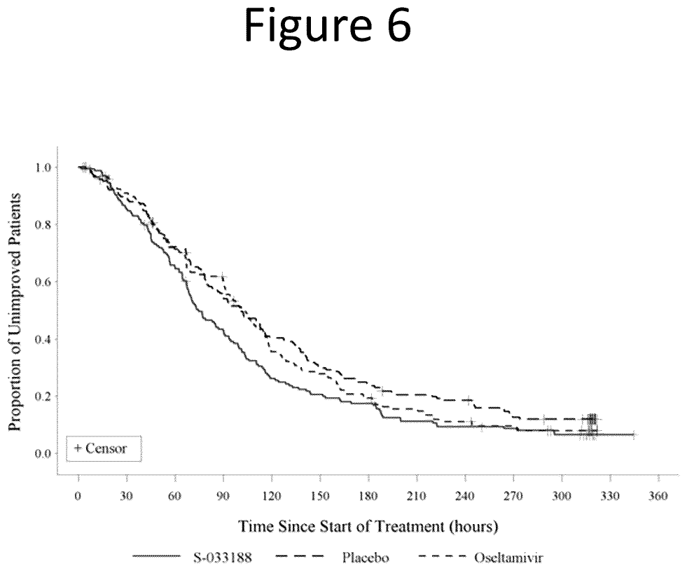

For patients infected with a type B influenza virus, the median time to improvement of influenza symptoms was statistically significantly shorter in the BXM group (74.6 hours [95% CI: 67.4, 90.2]) compared to the placebo group (100.6 hours [95% CI: 82.8, 115.8]) (median difference of −26.0 hours; generalized Wilcoxon test p-value =0.0138) and compared to the oseltamivir group (101.6 hours, median difference of −27.1 hours, generalized Wilcoxon test p-value =0.0251). The significance is realized in that the BXM and oseltamivir are equivalent in otherwise healthy patients. Details of the results for the type B influenza virus are shown in the table of FIG. 5 and the graph of FIG. 6.

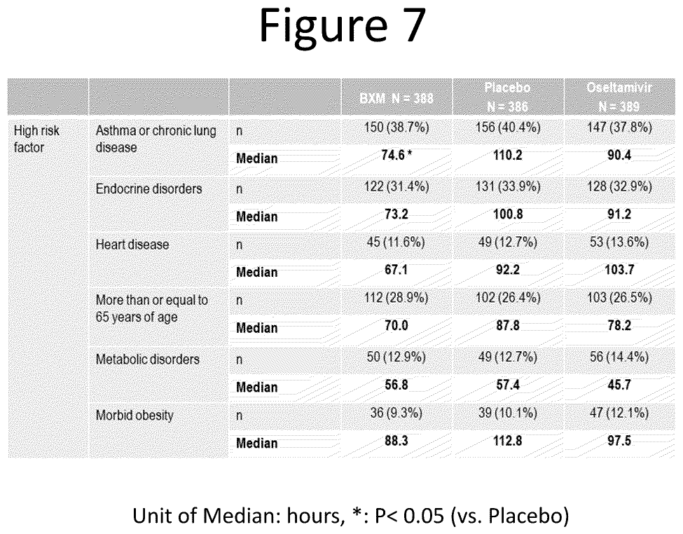

Details of the results for patients having a certain complication risk factor are provided in the table of FIG. 7.

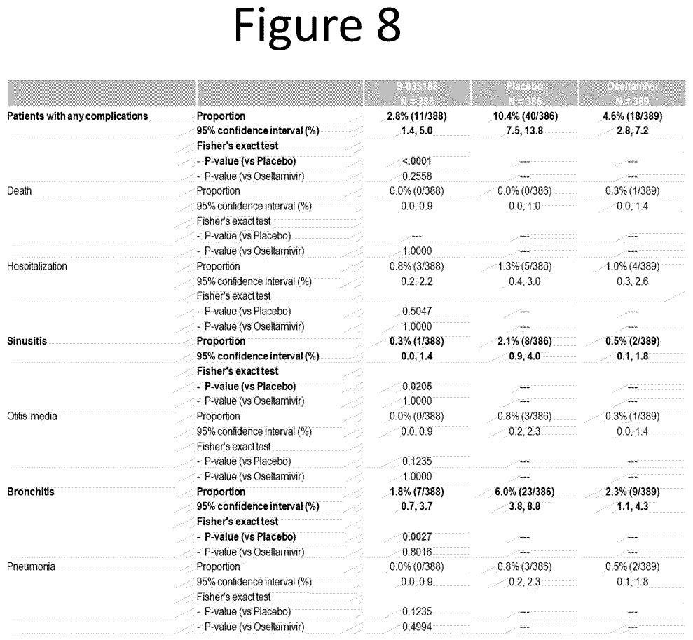

Details of the results for patients who experienced influenza-related complications (death, hospitalization, sinusitis, otitis media, bronchitis, pneumonia) are provided in the table of FIG. 8. BXM was statistically significantly superior to placebo for all patients with any complications. BXM was statistically significantly superior to placebo with respect to sinusitis and bronchitis.

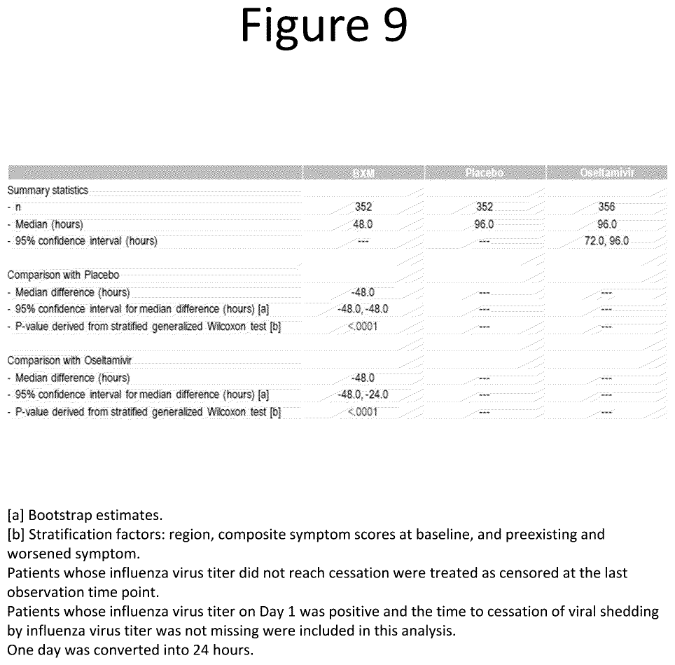

Details as to the results of the time to cessation of viral shedding by a virus titer are provided in FIG. 9. BXM was statistically significantly superior to placebo and oseltamivir.

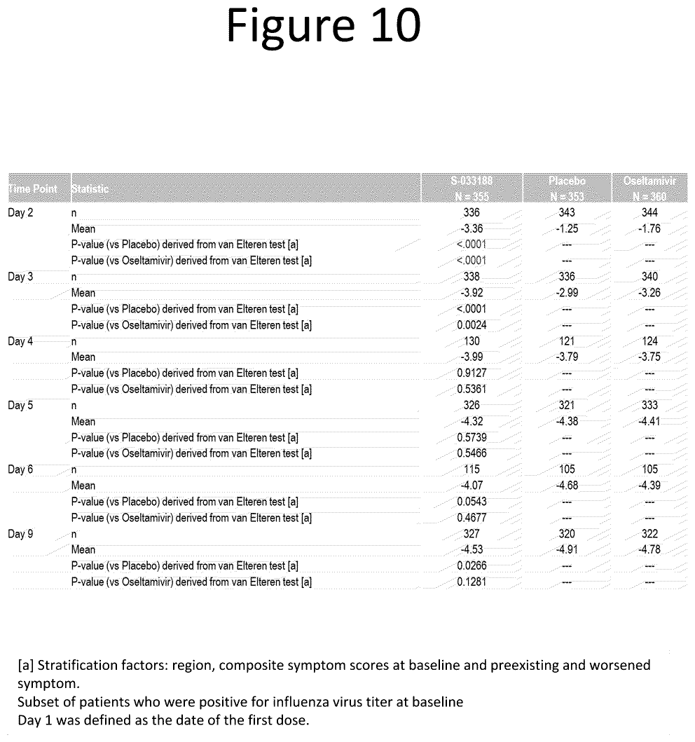

A summary of the statistical results of a change from baseline in an influenza virus titer [log10(TCID50/mL)] by time is provided in FIG. 10. BXM was statistically significantly superior to placebo and oseltamivir by day 2.

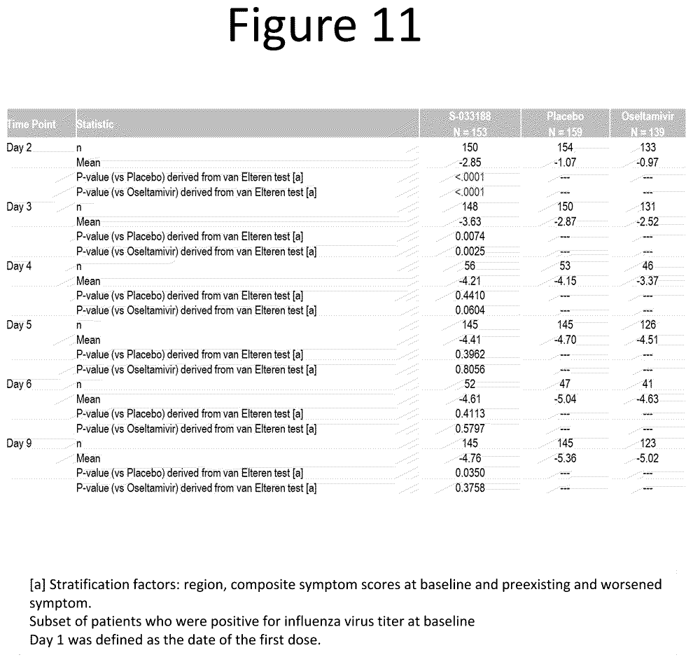

The statistical results for patients infected with type B virus are provided in FIG. 11. BXM was statistically significantly superior to placebo and oseltamivir by day 2.

As for the secondary efficacy endpoint, the efficacy and the side effects of the investigational drug were evaluated according to the influenza virus titer using a nasal or throat swab.

### Formulation Example

The following Formulation Examples only exemplify and are not intended to limit the scope of the invention.

### Formulation Example 1: Tablets

The compounds used in the present invention, lactose and calcium stearate are mixed. The mixture is crushed, granulated and dried to give a suitable size of granules. Next, calcium stearate is added to the granules, and the mixture is compressed and molded to give tablets.

### Formulation Example 2: Capsules

The compounds used in the present invention, lactose and calcium stearate are mixed uniformly to obtain powder medicines in the form of powders or fine granules. The powder medicines are filled into capsule containers to give capsules.

### Formulation Example 3: Granules

The compounds used in the present invention, lactose and calcium stearate are mixed uniformly and the mixture is compressed and molded. Then, it is crushed, granulated and sieved to give suitable sizes of granules.

### Formulation Example 4: Orally Disintegrated Tablets

The compounds used in the present invention and crystalline cellulose are mixed, granulated and tablets are made to give orally disintegrated tablets.

### Formulation Example 5: Dry Syrups

The compounds used in the present invention and lactose are mixed, crushed, granulated and sieved to give suitable sizes of dry syrups.

### Formulation Example 6: Injections

The compounds used in the present invention and phosphate buffer are mixed to give injection.

### Formulation Example 7: Infusions

The compounds used in the present invention and phosphate buffer are mixed to give injection.

### Formulation Example 8: Inhalations

The compound used in the present invention and lactose are mixed and crushed finely to give inhalations.

### Formulation Example 9: Ointments

The compounds used in the present invention and petrolatum are mixed to give ointments.

### Formulation Example 10: Patches

The compounds used in the present invention and base such as adhesive plaster or the like are mixed to give patches.

1-30. (canceled)

31. A method for reducing time to improvement of at least one symptom of an influenza virus infection in a human subject in need thereof, comprising:
administering an effective amount of a compound having one of following formula (I) or formula (II) to the human subject in need thereof: 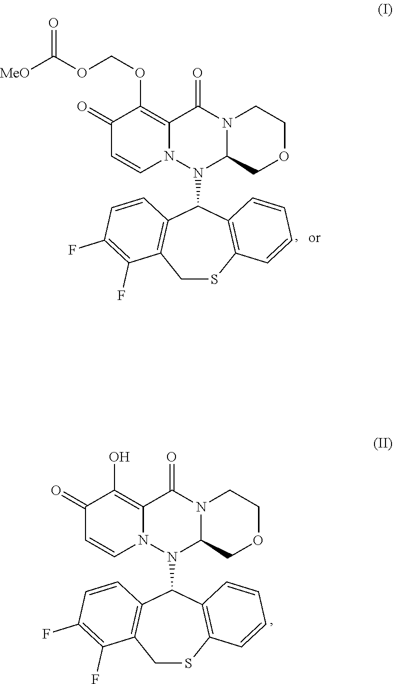 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]
or a pharmaceutically acceptable salt thereof, wherein:
the human subject has an influenza B virus infection;
the human subject is at a high risk of an influenza complication due to having at least one complication risk factor selected from the group consisting of chronic lung disease, an endocrine disorder, heart disease, morbid obesity, being 65 years of age or older, being a current resident of a long-term care facility, a compromised immune system, a neurological disorder, a neurodevelopmental disorder, a blood disorder, being a female who is within two weeks postpartum and is not breastfeeding, and having American Indian or Alaskan native heritage; and
the complication risk factor is not caused by the influenza B virus infection.

32. The method of claim 31, wherein the human subject has at least one complication risk factor selected from the group consisting of chronic lung disease, diabetes, heart disease, morbid obesity, and being 65 years of age or older.

33. The method of claim 31, wherein the reduction in the time to improvement of the at least one symptom of the influenza virus infection is relative to that of a human subject with an influenza B virus infection who is not treated for the influenza B virus infection and who has at least one complication risk factor independently selected from the group of the complication risk factors.

34. The method of claim 31, wherein the reduction in the time to improvement of the at least one symptom of the influenza virus infection is relative to that of a human subject with an influenza B virus infection who is treated with oseltamivir for the influenza B virus infection and who has at least one complication risk factor independently selected from the group of the complication risk factors.

35. The method of claim 31, wherein the at least one symptom is selected from the group consisting of headache, feverishness, chills, muscular pain, joint pain, fatigue, cough, sore throat, and nasal congestion.

36. The method of claim 31, wherein:
the human subject weighs in a range from 40 kg to less than 80 kg; and
the effective amount administered is about 40 mg of the compound of formula (I).

37. The method of claim 31, wherein:
the human subject weighs at least 80 kg; and
the effective amount administered is about 80 mg of the compound of formula (I).

38. The method of claim 31, wherein the time to improvement of the at least one symptom of the influenza virus infection is within 90.2 hours.

39. The method of claim 31, wherein the time to improvement of the at least one symptom of the influenza virus infection is within 74.6 hours.

40. The method of claim 31, wherein the time to improvement of the at least one symptom of the influenza virus infection is within 67.4 hours.

41. The method of claim 31, wherein the time to improvement of the at least one symptom of the influenza virus infection is reduced by at least 15.4 hours.

42. The method of claim 31, wherein the time to improvement of the at least one symptom of the influenza virus infection is reduced by at least 25.6 hours.

43. The method of claim 31, wherein the time to improvement of the at least one symptom of the influenza virus infection is reduced by at least 26 hours.

44. The method of claim 31, wherein the time to improvement of the at least one symptom of the influenza virus infection is reduced by at least 27.1 hours.

45. The method of claim 41, wherein the reduction in the time to improvement of the at least one symptom of the influenza virus infection is relative to that of a human subject with an influenza B virus infection who is not treated for the influenza B virus infection and who has at least one complication risk factor independently selected from the group of the complication risk factors.

46. The method of claim 42, wherein the reduction in the time to improvement of the at least one symptom of the influenza virus infection is relative to that of a human subject with an influenza B virus infection who is not treated for the influenza B virus infection and who has at least one complication risk factor independently selected from the group of the complication risk factors.

47. The method of claim 43, wherein the reduction in the time to improvement of the at least one symptom of the influenza virus infection is relative to that of a human subject with an influenza B virus infection who is not treated for the influenza B virus infection and who has at least one complication risk factor independently selected from the group of the complication risk factors.

48. The method of claim 44, wherein the reduction in the time to improvement of the at least one symptom of the influenza virus infection is relative to that of a human subject with an influenza B virus infection who is treated with oseltamivir for the influenza B virus infection and who has at least one complication risk factor independently selected from the group of the complication risk factors.

49. A method for reducing time to improvement of at least one symptom of an influenza virus infection in a human subject in need thereof, comprising:
administering an effective amount of a compound of formula (1) to the human subject in need thereof: 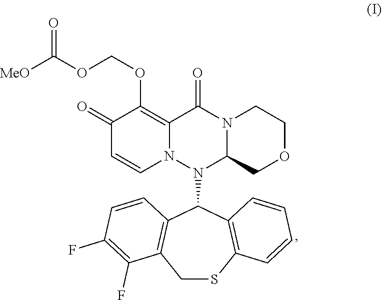 [SMILES: COC(=O)OCOc1c2n(ccc1=O)N([C@@H]1c3ccccc3SCc3c1ccc(F)c3F)[C@@H]1COCCN1C2=O]
wherein:
the human subject has an influenza B virus infection;
the human subject is at high risk of an influenza complication due to having at least one complication risk factor selected from the group consisting of chronic lung disease, an endocrine disorder, heart disease, morbid obesity, being 65 years of age or older, being a current resident of a long-term care facility, a compromised immune system, a neurological disorder, a neurodevelopmental disorder, a blood disorder, being a female who is within two weeks postpartum and is not breastfeeding, and having American Indian or Alaskan native heritage; and
the complication risk factor is not caused by the influenza B virus infection;
and wherein:
the human subject weighs in a range from 40 kg to less than 80 kg and the effective amount administered is about 40 mg of the compound of formula (I); or
the human subject weighs at least 80 kg and the effective amount administered is about 80 mg of the compound of formula (I).

50. The method of claim 49, wherein the time to improvement of the at least one symptom of the influenza virus infection is within 90.2 hours.

51. The method of claim 49, wherein the time to improvement of the at least one symptom of influenza virus infection is within 74.6 hours.

52. The method of claim 49, wherein the time to improvement of the at least one symptom of the influenza virus infection is within 67.4 hours.

53. The method of claim 49, wherein the time to improvement of the at least one symptom of the influenza virus infection is reduced by at least 15.4 hours, relative to that of a human subject with an influenza B virus infection who is not treated for the influenza B virus infection and who has at least one complication risk factor independently selected from the group of the complication risk factors.

54. The method of claim 49, wherein the time to improvement of the at least one symptom of the influenza virus infection is reduced by at least 25.6 hours, relative to that of a human subject with an influenza B virus infection who is not treated for the influenza B virus infection and who has at least one complication risk factor independently selected from the group of the complication risk factors.

55. The method of claim 49, wherein the time to improvement of the at least one symptom of the influenza virus infection is reduced by at least 26 hours, relative to that of a human subject with an influenza B virus infection who is not treated for the influenza B virus infection and who has at least one complication risk factor independently selected from the group of the complication risk factors.

56. The method of claim 49, wherein the time to improvement of the at least one symptom of the influenza virus infection is reduced by at least 27.1 hours, relative to that of a human subject with an influenza B virus infection who is treated with oseltamivir for the influenza B virus infection and who has at least one complication risk factor independently selected from the group of the complication risk factors.

57. The method of claim 49, wherein the human subject has at least one complication risk factor selected from the group consisting of chronic lung disease, diabetes, heart disease, morbid obesity, and being 65 years of age or older, and the at least one symptom is selected from the group consisting of headache, feverishness, chills, muscular pain, joint pain, fatigue, cough, sore throat, and nasal congestion.
